SEQUENCE LISTING (25 sequences)

SEQ ID NO: 1 - DNA, 2679 bp, Clostridium difficile
  CDS (1)..(2679)
acatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttaccccgggtaa

SEQ ID NO: 2 - protein, 892 aa, Clostridium difficile
TYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYPG

SEQ ID NO: 3 - DNA, 1635 bp, Clostridium difficile
  CDS (1)..(1635)
aagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgag

SEQ ID NO: 4 - protein, 545 aa, Clostridium difficile
KFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 5 - protein, 305 aa, Salmonella typhi
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAGAAAGIVAGPFGLIISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVAS

SEQ ID NO: 6 - protein, 305 aa, Artificial sequence
  Modified ClyA sequence
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAVAAAGSVSGPFGLSISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHISGKKTLFEVPDV

SEQ ID NO: 7 - DNA, 1102 bp, Salmonella typhi
ggaggtaataggtaagaatactttataaaacaggtacttaattgcaatttatatatttaaagaggcaaatgattatgaccggaatatttgcagaacaaactgtagaggtagttaaaagcgcgatcgaaaccgcagatggggcattagatctttataacaaatacctcgaccaggtcatcccctggaagacctttgatgaaaccataaaagagttaagccgttttaaacaggagtactcgcaggaagcttctgttttagttggtgatattaaagttttgcttatggacagccaggacaagtattttgaagcgacacaaactgtttatgaatggtgtggtgtcgtgacgcaattactctcagcgtatattttactatttgatgaatataatgagaaaaaagcatcagcccagaaagacattctcattaggatattagatgatggtgtcaagaaactgaatgaagcgcaaaaatctctcctgacaagttcacaaagtttcaacaacgcttccggaaaactgctggcattagatagccagttaactaatgatttttcggaaaaaagtagttatttccagtcacaggtggatagaattcgtaaggaagcttatgccggtgctgcagccggcatagtcgccggtccgtttggattaattatttcctattctattgctgcgggcgtgattgaagggaaattgattccagaattgaataacaggctaaaaacagtgcaaaatttctttactagcttatcagctacagtgaaacaagcgaataaagatatcgatgcggcaaaattgaaattagccactgaaatagcagcaattggggagataaaaacggaaaccgaaacaaccagattctacgttgattatgatgatttaatgctttctttattaaaaggagctgcaaagaaaatgattaacacctgtaatgaataccaacaaagacacggtaagaagacgcttttcgaggttcctgacgtctgatacattttcattcgatctgtgtacttttaacgcccgatagcgtaaagaaaatgagagacggagaaaaagcgatattcaacagcccgataaacaagagtcgttaccgggctgacgag

SEQ ID NO: 8 - DNA, 1102 bp, Salmonella paratyphi
ggaggcaataggtaggaataagttataaaacaatagcttaattgcaatttatatatttaaagaggcaaatgattatgactggaatatttgcagaacaaactgtagaggtagttaaaagcgcgatcgaaaccgcagatggggcattagatttttataacaaatacctcgaccaggttatcccctggaagacctttgatgaaaccataaaagagttaagccgttttaaacaggagtactcgcaggaagcttctgttttagttggtgatattaaagttttgcttatggacagccaggataagtattttgaagcgacacaaactgtttatgaatggtgtggtgtcgtgacgcaattactctcagcgtatattttactatttgatgaatataatgagaaaaaagcatcagcgcagaaagacattctcatcaggatattagatgatggcgtcaataaactgaatgaagcgcaaaaatctctcctgggaagttcacaaagtttcaacaacgcttcaggaaaactgctggcattagatagccagttaactaatgatttctcggaaaaaagtagttatttccagtcacaggtggatagaattcgtaaggaagcttatgccggtgctgcagcaggcatagtcgccggtccgtttggattaattatttcctattctattgctgcgggcgtgattgaagggaaattgattccagaattgaatgacaggctaaaagcagtgcaaaatttctttactagcttatcagtcacagtgaaacaagcgaataaagatatcgatgcggcaaaattgaaattagccactgaaatagcagcaattggggagataaaaacggaaaccgaaacaaccagattctacgttgattatgatgatttaatgctttctttactaaaaggagctgcaaagaaaatgattaacacctgtaatgaataccaacaaaggcacggtaagaagacgcttctcgaggttcctgacatctgatacattttcattcgctctgtttacttttaacgcccgatagcgtgaagaaaatgagagacggagaaaaagcgatattcaacagcccgataaacaagagtcgttaccgggctggcgag

SEQ ID NO: 9 - DNA, 904 bp, Shigella flexneri
atgactgaaatcgttgcagataaaacggtagaagtagttaaaaacgcaatcgaaaccgcagatggagcattagatctttataataaatatctcgatcaggtcatcccctggcagacctttgatgaaaccataaaagagttaagtcgctttaaacaggagtattcacaggcagcctccgttttagtcggcgatattaaaaccttacttatggatagccaggataagtattttgaagcaacccaaacagtgtatgaatggtgtggtgttgcgacgcaattgctcgcagcgtatattttgctatttgatgagtacaatgagaagaaagcatccgcccctcattaaggtactggatgacggcatcacgaagctgaatgaagcgcaaaattccctgctggtaagctcacaaagtttcaacaacgcttccgggaaactgctggcgttagatagccagttaaccaatgatttttcagaaaaaagcagctatttccagtcacaggtagataaaatcaggaaggaagcgtatgccggtgccgcagccggtgtcgtcgccggtccatttggtttaatcatttcctattctattgctgcgggcgtagttgaagggaaactgattccagaattgaagaacaagttaaaatctgtgcagagtttctttaccaccctgtctaacacggttaaacaagcgaataaagatatcgatgccgccaaattgaaattaaccaccgaaatagccgccatcggggagataaaaacggaaactgaaaccaccagattctatgttgattatgatgatttaatgctttctttgctaaaagcagcggccaaaaaaatgattaacacctgtaatgagtatcagaaaagacacggtaaaaagacactctttgaggtacctgaagtctgataa

SEQ ID NO: 10 - DNA, 1080 bp, Escherichia coli
agaaataaagacattgacgcatcccgcccggctaactatgaattagatgaagtaaaatttattaatagttgtaaaacaggagtttcattacaatttatatatttaaagaggcgaatgattatgactgaaatcgttgcagataaaacggtagaagtagttaaaaacgcaatcgaaaccgcagatggagcattagatctttataataaatatctcgatcaggtcatcccctggcagacctttgatgaaaccataaaagagttaagtcgctttaaacaggagtattcacaggcagcctccgttttagtcggcgatattaaaaccttacttatggatagccaggataagtattttgaagcaacccaaacagtgtatgaatggtgtggtgttgcgacgcaattgctcgcagcgtatattttgctatttgatgagtacaatgagaagaaagcatccgcccagaaagacattctcattaaggtactggatgacggcatcacgaagctgaatgaagcgcaaaaatccctgctggtaagctcacaaagtttcaacaacgcttccgggaaactgctggcgttagatagccagttaaccaatgatttttcagaaaaaagcagctatttccagtcacaggtagataaaatcaggaaggaagcatatgccggtgccgcagccggtgtcgtcgccggtccatttggattaatcatttcctattctattgctgcgggcgtagttgaaggaaaactgattccagaattgaagaacaagttaaaatctgtgcagaatttctttaccaccctgtctaacacggttaaacaagcgaataaagatatcgatgccgccaaattgaaattaaccaccgaaatagccgccatcggtgagataaaaacggaaactgaaacaaccagattctacgttgattatgatgatttaatgctttctttgctaaaagaagcggccaaaaaaatgattaacacctgtaatgagtatcagaaaagacacggtaaaaagacactctttgaggtacctgaagtctgataagcgattattctctccatgtactcaaggtataaggtttatcacatt

SEQ ID NO: 11 - protein, 50 aa, Salmonella typhi
MSFSRRQFLQASGIALCAGAIPLRANAAGQQQPLPVPPLLESRRGQPLFM

SEQ ID NO: 12 - DNA, 3594 bp, Artificial Sequence
  Fusion A Codon-optimized sequence
atgacttcgatcttcgccgaacagacggttgaggtggtaaaatcagccatagaaaccgcggatggggcgctcgacctttacaataagtaccttgatcaggtgatcccgtggaaaacgttcgacgagactatcaaagaattatcacgatttaagcaggaatattcacaggaagcatccgtacttgttggtgatattaaagtcttactcatggattctcaggataagtacttcgaggcaacccagacggtgtacgagtggtgtggcgttgtaacacagcttctgtcggcttacattcttctgttcgatgaatataacgagaaaaaagcctccgcccagaaagacattctgatacgcattcttgacgatggtgtgaagaagctgaacgaagcacagaaatcgttattaacttcctctcagtcctttaataacgcgtcaggcaagttactggctcttgattcccagttgactaatgacttcagtgaaaaatcgtcgtatttccagtcacaagttgaccgtatccgtaaagaggcttacgctgtcgctgctgcgggctcggtcagtggcccattcggtctttctatcagctatagcattgcagccggagtcatagaaggcaaactgatcccggagttgaacaatcgcctgaaaaccgtgcaaaatttttttacgagtttgagcgccactgtcaaacaggcgaacaaggatatagatgctgcaaaactcaaattagcgaccgaaattgccgcgataggtgaaattaagaccgaaacggagacaacccggttctacgtcgactacgacgacttgatgttatcattgctgaaaggcgccgctaaaaagatgatcaacacctgtaacgaatatcagcagcggcacggaaaaaaaaccctttttgaggtccctgatgtcgggcccacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttaccccgggtaa

SEQ ID NO: 13 - protein, 1197 aa, Artificial Sequence
  Synthetic Construct
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAVAAAGSVSGPFGLSISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVGPTYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYPG

SEQ ID NO: 14 - DNA, 2550 bp, Artificial Sequence
  Fusion B Codon-optimized sequence
atgaccagcattttcgccgaacagactgtggaagtggtgaagtcggcaatcgaaaccgcggacggcgctctggatctgtataacaaatatctggaccaggtaatcccctggaaaaccttcgatgaaacgatcaaagaactttcgaggtttaagcaggaatattcgcaggaagcctcagtcctcgtcggcgatatcaaagtgctgctcatggattctcaggataagtatttcgaagcaacgcagacggtctatgaatggtgtggggtggtcacacagttactttccgcatacatccttctgttcgatgaatacaacgaaaaaaaggcatccgcgcagaaagatatcttaatcaggattcttgatgacggtgttaagaaactgaacgaagctcagaaatcgctgcttacaagctcccagtcgttcaacaatgcgtcaggtaaactgttagcgcttgactcacagttgacaaatgatttctctgaaaagagcagttatttccagtcccaggtggatagaataagaaaagaggcatacgcggtggcagccgctggttcggtgtccgggccattcggtctgtcgatttcttatagcattgcggctggtgttatcgagggaaagctgattccggagcttaataaccgacttaagaccgtgcagaacttctttacttcactcagcgcgacagtcaagcaggccaacaaggatatcgacgccgccaaactcaagctggccacagaaattgctgcaatcggtgagataaagacagagacagaaacgacccgcttctatgtggactatgatgaccttatgttgagtctccttaaaggagccgccaaaaagatgataaacacgtgcaacgagtatcaacaaaggcatggaaaaaagacattatttgaagttccagacgttcccgggaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgag

SEQ ID NO: 15 - protein, 850 aa, Artificial Sequence
  Synthetic Construct
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAVAAAGSVSGPFGLSISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVPGKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 16 - DNA, 506 bp, Salmonella sp.
gcgcgccgctcgtagccctggcagggattggccttgctattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagcatt

SEQ ID NO: 17 - DNA, 9290 bp, Artificial Sequence
  ssaG antigen operon
tatccgaacggtcaaaacggatttttcgtattctcccgccgcgtcaatgctgatttatccctgtcttcgtggcaaactagccgccgaatttaatgcgagcatgccctggaggaatacgtggataaaattttcgtcgatgaagcagtaagtgaactgcataccattcaggacatgttgcgctgggcggtaagccgctttagcgcggcgaatatctggtatggacacggtaccgataacccgtgggatgaagcggtacaactggtgttgccgtctctttatctgccgctggatattccggaggatatgcggaccgcgcggctgacgtccagcgaaagacaccgcattgtcgagcgagtgattcgtcgcattaacgagcgtatcccggtagcctacctgaccaataaagcctggttctgcggccacgaattttatgttgatgagcgcgtgctggtgccgcgttcaccgattggcgagctgattaataaccacttcgctggccttattagccaacagccgaaatatattctggatatgtgtaccggcagcggctgcatcgccatcgcctgtgcttatgctttcccggacgcagaggttgatgcggtcgatatttcgccggatgcgctggctgtcgccgagcataacattgaagaacacggtcttatccatcacgtgacgccaatccgttccgatctgttccgcgatctgccgaaagttcagtacgatctgattgtcactaacccgccttatgtcgatgcggaggatatgtccgatctgccgaacgaatatcgccacgaacctgagctggggctggcgtccggcactgacggcctcaaattgacccgccgtatcctgggaaatgcgccggattatctgtccgatgatggcgttctgatttgtgaagtcggaaacagcatggtacatctgatggagcagtatccggatgtgccgttcacctggctggagtttgacaacggcggcgatggcgtctttatgttgaccaaagcgcagttgctcgcggcccgtgaacatttcaatatttataaagattaaaacacgcaaacgacaacaacgataacggagccgtgatggcaggaaacacaattggacaactctttcgcgtaaccactttcggcgaatcacacgggctggcgcttgggggtatcgtcgatggcgtgccgcccggcatcccgttgacggaggccgatctgcagcacgatctcgacagacgccgccctggcacctcgcgctatactactcagcgccgcgaaccggaccaggtaaaaattctctccggcgtgtttgatggcgtgacgaccggctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgacttcgatcttcgccgaacagacggttgaggtggtaaaatcagccatagaaaccgcggatggggcgctcgacctttacaataagtaccttgatcaggtgatcccgtggaaaacgttcgacgagactatcaaagaattatcacgatttaagcaggaatattcacaggaagcatccgtacttgttggtgatattaaagtcttactcatggattctcaggataagtacttcgaggcaacccagacggtgtacgagtggtgtggcgttgtaacacagcttctgtcggcttacattcttctgttcgatgaatataacgagaaaaaagcctccgcccagaaagacattctgatacgcattcttgacgatggtgtgaagaagctgaacgaagcacagaaatcgttattaacttcctctcagtcctttaataacgcgtcaggcaagttactggctcttgattcccagttgactaatgacttcagtgaaaaatcgtcgtatttccagtcacaagttgaccgtatccgtaaagaggcttacgctgtcgctgctgcgggctcggtcagtggcccattcggtctttctatcagctatagcattgcagccggagtcatagaaggcaaactgatcccggagttgaacaatcgcctgaaaaccgtgcaaaatttttttacgagtttgagcgccactgtcaaacaggcgaacaaggatatagatgctgcaaaactcaaattagcgaccgaaattgccgcgataggtgaaattaagaccgaaacggagacaacccggttctacgtcgactacgacgacttgatgttatcattgctgaaaggcgccgctaaaaagatgatcaacacctgtaacgaatatcagcagcggcacggaaaaaaaaccctttttgaggtccctgatgtcgggcccacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttaccccgggtaaggaaaagccatatgaccagcattttcgccgaacagactgtggaagtggtgaagtcggcaatcgaaaccgcggacggcgctctggatctgtataacaaatatctggaccaggtaatcccctggaaaaccttcgatgaaacgatcaaagaactttcgaggtttaagcaggaatattcgcaggaagcctcagtcctcgtcggcgatatcaaagtgctgctcatggattctcaggataagtatttcgaagcaacgcagacggtctatgaatggtgtggggtggtcacacagttactttccgcatacatccttctgttcgatgaatacaacgaaaaaaaggcatccgcgcagaaagatatcttaatcaggattcttgatgacggtgttaagaaactgaacgaagctcagaaatcgctgcttacaagctcccagtcgttcaacaatgcgtcaggtaaactgttagcgcttgactcacagttgacaaatgatttctctgaaaagagcagttatttccagtcccaggtggatagaataagaaaagaggcatacgcggtggcagccgctggttcggtgtccgggccattcggtctgtcgatttcttatagcattgcggctggtgttatcgagggaaagctgattccggagcttaataaccgacttaagaccgtgcagaacttctttacttcactcagcgcgacagtcaagcaggccaacaaggatatcgacgccgccaaactcaagctggccacagaaattgctgcaatcggtgagataaagacagagacagaaacgacccgcttctatgtggactatgatgaccttatgttgagtctccttaaaggagccgccaaaaagatgataaacacgtgcaacgagtatcaacaaaggcatggaaaaaagacattatttgaagttccagacgttcccgggaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgagtaatctagactagcctaggtccagcattaccgtgccgggacgtacgatcaaccggatgggtgaagaggtcgagatgatcaccaaagggcgccacgatccgtgtgtggggattcgcgcagtgccgatcgcagaagccatgctggcgatcgtactgatggatcacctgctgcgccatcgggcacagaatgcggatgtaaagacagagattccacgctggtaagaaatgaaaaaaaccgcgattgcgctgctggcatggtttgtcagtagcgccagcctggcggcgacgccgtggcagaaaataacccatcctgtccccggcgccgcccagtctatcggtagctttgccaacggatgcatcattggcgccgacacgttgccggtacagtccgataattatcaggtgatgcgcaccgatcagcgccgttatttcggccacccggatctggtcatgtttatccagcggttgagtcatcaggcgcagcaacgggggctcggaaccgtcctgataggcgacatggggatgcctgccggaggccgctttaatggcggacacgccagtcatcagaccgggcttgatgtggatattttcttgcagttgccgaaaacgcgctggagccaggcgcagctattgcgcccgcaggcgttagatctggtgtcccgcgacggtaaacatgtcgtgccgtcgcgctggtcgtcggatatcgccagtctgatcaaactggcggcacaagacaatgacgtcacccgtattttcgtcaatccggctattaaacaacagctttgcctcgatgccggaagcgatcgtgactggctacgtaaagtacgcccctggttccagcatcgcgcgcatatgcacgtgcgtttacgctgccctgccgacagcctggagtgcgaagatcaacctttacccccgccgggcgatggatgcggcgctgaactgcaaagctggttcgaaccgccaaaacctggcaccacaaagcctgagaagaagacaccgccgccgttgccgccttcctgccaggcgctactggatgagcatgtactctgatggacaatttttatgatctgtttatggtctccccgctgctgctggtggtgctgttttttgtcgccgtactggcaggatttatcgattctatcgccggaggcggagggctgctcactatccctgcgctgatggccgccgggatgtcgccggcaaacgcgttggcgaccaataaattacaggcgtgcggcggctccctctcgtcttcgctctattttattcgccgtaaagtggtaaacctggccgagcaaaagctcaatattctgatgacgttcattggctcgatgagcggcgcgct

SEQ ID NO: 18 - protein, 1197 aa, Artificial Sequence; identical to SEQ ID NO: 13
  ClyA-Toxin A repeat fusion sequence

SEQ ID NO: 19 - protein, 850 aa, Artificial Sequence; identical to SEQ ID NO: 15
  ClyA-Toxin B repeat fusion sequence

SEQ ID NO: 20 - DNA, 8356 bp, Artificial Sequence
  ClyA-Toxin A repeats-Toxin B repeats in aroC and under the control of an ssaG promoter
aacggtcaaaacggatttttcgtattctcccgccgcgtcaatgctgatttatccctgtcttcgtggcaaactagccgccgaatttaatgcgagcatgccctggaggaatacgtggataaaattttcgtcgatgaagcagtaagtgaactgcataccattcaggacatgttgcgctgggcggtaagccgctttagcgcggcgaatatctggtatggacacggtaccgataacccgtgggatgaagcggtacaactggtgttgccgtctctttatctgccgctggatattccggaggatatgcggaccgcgcggctgacgtccagcgaaagacaccgcattgtcgagcgagtgattcgtcgcattaacgagcgtatcccggtagcctacctgaccaataaagcctggttctgcggccacgaattttatgttgatgagcgcgtgctggtgccgcgttcaccgattggcgagctgattaataaccacttcgctggccttattagccaacagccgaaatatattctggatatgtgtaccggcagcggctgcatcgccatcgcctgtgcttatgctttcccggacgcagaggttgatgcggtcgatatttcgccggatgcgctggctgtcgccgagcataacattgaagaacacggtcttatccatcacgtgacgccaatccgttccgatctgttccgcgatctgccgaaagttcagtacgatctgattgtcactaacccgccttatgtcgatgcggaggatatgtccgatctgccgaacgaatatcgccacgaacctgagctggggctggcgtccggcactgacggcctcaaattgacccgccgtatcctgggaaatgcgccggattatctgtccgatgatggcgttctgatttgtgaagtcggaaacagcatggtacatctgatggagcagtatccggatgtgccgttcacctggctggagtttgacaacggcggcgatggcgtctttatgttgaccaaagcgcagttgctcgcggcccgtgaacatttcaatatttataaagattaaaacacgcaaacgacaacaacgataacggagccgtgatggcaggaaacacaattggacaactctttcgcgtaaccactttcggcgaatcacacgggctggcgcttgggggtatcgtcgatggcgtgccgcccggcatcccgttgacggaggccgatctgcagcacgatctcgacagacgccgccctggcacctcgcgctatactactcagcgccgcgaaccggaccaggtaaaaattctctccggcgtgtttgatggcgtgacgaccggctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgacttcgatcttcgccgaacagacggttgaggtggtaaaatcagccatagaaaccgcggatggggcgctcgacctttacaataagtaccttgatcaggtgatcccgtggaaaacgttcgacgagactatcaaagaattatcacgatttaagcaggaatattcacaggaagcatccgtacttgttggtgatattaaagtcttactcatggattctcaggataagtacttcgaggcaacccagacggtgtacgagtggtgtggcgttgtaacacagcttctgtcggcttacattcttctgttcgatgaatataacgagaaaaaagcctccgcccagaaagacattctgatacgcattcttgacgatggtgtgaagaagctgaacgaagcacagaaatcgttattaacttcctctcagtcctttaataacgcgtcaggcaagttactggctcttgattcccagttgactaatgacttcagtgaaaaatcgtcgtatttccagtcacaagttgaccgtatccgtaaagaggcttacgctgtcgctgctgcgggctcggtcagtggcccattcggtctttctatcagctatagcattgcagccggagtcatagaaggcaaactgatcccggagttgaacaatcgcctgaaaaccgtgcaaaatttttttacgagtttgagcgccactgtcaaacaggcgaacaaggatatagatgctgcaaaactcaaattagcgaccgaaattgccgcgataggtgaaattaagaccgaaacggagacaacccggttctacgtcgactacgacgacttgatgttatcattgctgaaaggcgccgctaaaaagatgatcaacacctgtaacgaatatcagcagcggcacggaaaaaaaaccctttttgaggtccctgatgtcgggcccacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttaccccgggaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgagtaatctagactagcctaggtccagcattaccgtgccgggacgtacgatcaaccggatgggtgaagaggtcgagatgatcaccaaagggcgccacgatccgtgtgtggggattcgcgcagtgccgatcgcagaagccatgctggcgatcgtactgatggatcacctgctgcgccatcgggcacagaatgcggatgtaaagacagagattccacgctggtaagaaatgaaaaaaaccgcgattgcgctgctggcatggtttgtcagtagcgccagcctggcggcgacgccgtggcagaaaataacccatcctgtccccggcgccgcccagtctatcggtagctttgccaacggatgcatcattggcgccgacacgttgccggtacagtccgataattatcaggtgatgcgcaccgatcagcgccgttatttcggccacccggatctggtcatgtttatccagcggttgagtcatcaggcgcagcaacgggggctcggaaccgtcctgataggcgacatggggatgcctgccggaggccgctttaatggcggacacgccagtcatcagaccgggcttgatgtggatattttcttgcagttgccgaaaacgcgctggagccaggcgcagctattgcgcccgcaggcgttagatctggtgtcccgcgacggtaaacatgtcgtgccgtcgcgctggtcgtcggatatcgccagtctgatcaaactggcggcacaagacaatgacgtcacccgtattttcgtcaatccggctattaaacaacagctttgcctcgatgccggaagcgatcgtgactggctacgtaaagtacgcccctggttccagcatcgcgcgcatatgcacgtgcgtttacgctgccctgccgacagcctggagtgcgaagatcaacctttacccccgccgggcgatggatgcggcgctgaactgcaaagctggttcgaaccgccaaaacctggcaccacaaagcctgagaagaagacaccgccgccgttgccgccttcctgccaggcgctactggatgagcatgtactctgatggacaatttttatgatctgtttatggtctccccgctgctgctggtggtgctgttttttgtcgccgtactggcaggatttatcgattctatcgccggaggcggagggctgctcactatccctgcgctgatggccgccgggatgtcgccggcaaacgcgttggcgaccaataaattacaggcgtgcggcggctccctctcgtcttcgctctattttattcgccgtaaagtggtaaacctggccgagcaaaagctcaatattctgatgacgttcattggctcgatgagcggcgcgctg

SEQ ID NO: 21 - protein, 1742 aa, Artificial Sequence
  ClyA-Toxin A repeats-Toxin B repeats
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAVAAAGSVSGPFGLSISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVGPTYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYPGKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 22 - DNA, 7016 bp, Artificial Sequence
  ClyA-Toxin A repeat aroC and under the control of an ssaG promoter
cgcccccagttcctgcttggcctgaagctgcgttaagccgtgtaaatccagaaacagttctggcgaatagtcgccgcggcgcattttttttagctcgaaatggctgacatcttcacggacatactttaccggcccttccgtattgagcagcggctggaactcatcagaaaaatagtggctggcgtccgcctgttcctgaattaaccttcgggtagggacttccgtgattttcttgcgcagtggccgatggacgatagtatcctgtttaatctttcgggtgccgaccatcagctgacggaacagcgcctggtcctcctcgctgagcgatgttttctttttcattcgtgggtctcgtctgatcttttgccttagtttacccgacccggaggatatccgaacggtcaaaacggatttttcgtattctcccgccgcgtcaatgctgatttatccctgtcttcgtggcaaactagccgccgaatttaatgcgagcatgccctggaggaatacgtggataaaattttcgtcgatgaagcagtaagtgaactgcataccattcaggacatgttgcgctgggcggtaagccgctttagcgcggcgaatatctggtatggacacggtaccgataacccgtgggatgaagcggtacaactggtgttgccgtctctttatctgccgctggatattccggaggatatgcggaccgcgcggctgacgtccagcgaaagacaccgcattgtcgagcgagtgattcgtcgcattaacgagcgtatcccggtagcctacctgaccaataaagcctggttctgcggccacgaattttatgttgatgagcgcgtgctggtgccgcgttcaccgattggcgagctgattaataaccacttcgctggccttattagccaacagccgaaatatattctggatatgtgtaccggcagcggctgcatcgccatcgcctgtgcttatgctttcccggacgcagaggttgatgcggtcgatatttcgccggatgcgctggctgtcgccgagcataacattgaagaacacggtcttatccatcacgtgacgccaatccgttccgatctgttccgcgatctgccgaaagttcagtacgatctgattgtcactaacccgccttatgtcgatgcggaggatatgtccgatctgccgaacgaatatcgccacgaacctgagctggggctggcgtccggcactgacggcctcaaattgacccgccgtatcctgggaaatgcgccggattatctgtccgatgatggcgttctgatttgtgaagtcggaaacagcatggtacatctgatggagcagtatccggatgtgccgttcacctggctggagtttgacaacggcggcgatggcgtctttatgttgaccaaagcgcagttgctcgcggcccgtgaacatttcaatatttataaagattaaaacacgcaaacgacaacaacgataacggagccgtgatggcaggaaacacaattggacaactctttcgcgtaaccactttcggcgaatcacacgggctggcgcttgggggtatcgtcgatggcgtgccgcccggcatcccgttgacggaggccgatctgcagcacgatctcgacagacgccgccctggcacctcgcgctatactactcagcgccgcgaaccggaccaggtaaaaattctctccggcgtgtttgatggcgtgacgaccggctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgacttcgatcttcgccgaacagacggttgaggtggtaaaatcagccatagaaaccgcggatggggcgctcgacctttacaataagtaccttgatcaggtgatcccgtggaaaacgttcgacgagactatcaaagaattatcacgatttaagcaggaatattcacaggaagcatccgtacttgttggtgatattaaagtcttactcatggattctcaggataagtacttcgaggcaacccagacggtgtacgagtggtgtggcgttgtaacacagcttctgtcggcttacattcttctgttcgatgaatataacgagaaaaaagcctccgcccagaaagacattctgatacgcattcttgacgatggtgtgaagaagctgaacgaagcacagaaatcgttattaacttcctctcagtcctttaataacgcgtcaggcaagttactggctcttgattcccagttgactaatgacttcagtgaaaaatcgtcgtatttccagtcacaagttgaccgtatccgtaaagaggcttacgctgtcgctgctgcgggctcggtcagtggcccattcggtctttctatcagctatagcattgcagccggagtcatagaaggcaaactgatcccggagttgaacaatcgcctgaaaaccgtgcaaaatttttttacgagtttgagcgccactgtcaaacaggcgaacaaggatatagatgctgcaaaactcaaattagcgaccgaaattgccgcgataggtgaaattaagaccgaaacggagacaacccggttctacgtcgactacgacgacttgatgttatcattgctgaaaggcgccgctaaaaagatgatcaacacctgtaacgaatatcagcagcggcacggaaaaaaaaccctttttgaggtccctgatgtcgggcccacatattactacgacgaagattcgaagttggtcaagggcctgataaacataaacaactcgttattttatttcgatcctattgaatttaacctggtgacggggtggcagaccataaacgggaagaagtactactttgacatcaataccggcgcagcattgatttcatataagataattaacggcaagcatttctactttaacaacgatggagtcatgcaactgggagtctttaagggtcccgacggcttcgaatactttgccccagcgaacacccaaaacaacaatattgaggggcaggcgattgtctatcaatcaaagtttttgacgctgaacggtaagaaatactattttgataacgattcgaaagcagtcacggggtggcggattattaacaacgaaaaatattattttaatccaaataatgctatcgcagcagtcgggcttcaagtgatcgataataataagtactacttcaatccagatacggctattatttcaaaagggtggcagactgtcaacggctccaggtattatttcgacactgatactgctatcgctttcaacgggtataagacaatcgatggtaagcatttctactttgatagcgactgcgtggttaaaattggtgtattcagtacctctaatggatttgagtacttcgctcctgcaaacacttacaataacaatattgaaggtcaggccatcgtataccaaagcaagttcctcaccttaaatggcaaaaagtactatttcgacaacaatagcaaagcggtcaccggttggcagaccattgatagtaaaaaatattattttaataccaacactgcggaagctgctaccggatggcagacaatcgacggcaagaagtattatttcaacaccaatacagcagaagcggccacagggtggcaaacgatcgacgggaagaagtactactttaatactaacacggccattgctagcaccggttataccattattaatgggaaacacttttacttcaacactgacggcattatgcagatcggtgtattcaaagggcctaacggcttcgaatatttcgcaccggccaatacagacgcgaacaatatagaaggacaggcgattctgtatcagaatgaattcctgaccctgaatggtaagaaatattacttcggcagcgattctaaggccgtcaccgggtggcggataatcaataataaaaagtactatttcaacccgaataacgcgattgcagctattcacctgtgcacgatcaacaatgataagtattattttagctatgatgggatccttcaaaatggatatattacaatagaaagaaataacttctatttcgatgcgaataatgagtctaaaatggtgactggcgttttcaaaggcccaaatgggttcgaatacttcgctccggcgaacacacacaacaacaatattgaagggcaggcaatagtgtatcagaataaattcttgacgctgaatggtaaaaagtactactttgataatgattcgaaagcggtaacaggctggcagaccatagacggcaagaaatattactttaatctgaatactgccgaagctgcgacgggctggcaaaccatagacggaaagaaatattattttaatctgaacaccgcagaggccgccaccggatggcagaccatcgacgggaagaaatactatttcaacactaataccttcatagcgagtacggggtatacctcgatcaatggcaagcatttctactttaacaccgacgggattatgcagatcggtgttttcaaggggccgaacggcttcgaatacttcgctcccgcaaacacacacaacaacaacatcgagggacaggctatactgtatcaaaataaatttcttacgttaaatggcaagaagtattattttgggtcggacagcaaagcagtgaccggtttgcgtaccatagatggtaagaaatattattttaatactaacacggcagtagccgttaccggatggcagactattaatgggaagaaatactattttaacactaacacgagcattgcctcgactggctacacgatcattagcgggaaacacttctacttcaacacggatggtattatgcagataggtgtctttaaaggtcctgacggttttgagtacttcgcacccgccaacaccgacgctaataacatagaggggcaagctatcaggtatcagaatcgcttcctttacctgcatgataacatctattacttcgggaacaacagtaaggctgctaccgggtgggtgacaattgacggtaatcgctattatttcgagcctaacacagcaatgggagccaatggctataagactatcgataacaaaaatttttactttcggaacggtttgcctcaaatcggggtttttaaaggatctaacggcttcgagtactttgccccggcgaacacggatgccaacaatattgagggccaggcgataaggtaccagaaccgctttctgcatctcttgggtaaaatctattacttcggcaacaactcaaaggcggtaacaggatggcaaactataaacgggaaggtttactattttatgcctgatacggccatggctgcggcgggaggcctgttcgaaattgacggtgttatatactttttcggtgtggacggtgttaaggccccaggcatttacccggctagactagcctaggtccagcattaccgtgccgggacgtacgatcaaccggatgggtgaagaggtcgagatgatcaccaaagggcgccacgatccgtgtgtggggattcgcgcagtgccgatcgcagaagccatgctggcgatcgtactgatggatcacctgctgcgccatcgggcacagaatgcggatgtaaagacagagattccacgctggtaagaaatgaaaaaaaccgcgattgcgctgctggcatggtttgtcagtagcgccagcctggcggcgacgccgtggcagaaaataacccatcctgtccccggcgccgcccagtctatcggtagctttgccaacggatgcatcattggcgccgacacgttgccggtacagtccgataattatcaggtgatgcgcaccgatcagcgccgttatttcggccacccggatctggtcatgtttatccagcggttgagtcatcaggcgcagcaacgggggctcggaaccgtcctgataggcgacatggggatgcctgccggaggccgctttaatggcggacacgccagtcatcagaccgggcttgatgtggatattttcttgcagttgccgaaaacgcgctggagccaggcgcagctattgcgcccgcaggcgttagatctggtgtcccgcgacggtaaacatgtcgtgccgtcgcgctggtcgtcggatatcgccagtctgatcaaactggcggcacaagacaatgacgtcacccgtattttcgtcaatccggctattaaacaacagctttgcctcgatgccggaagcgatcgtgactggctacgtaaagtacgcccctggttccagcatcgcgcgcatatgcacgtgcgtttacgctgccctgccgacagcctggagtgcgaagatcaacctttacccccgccgggcgatggatgcggcgctgaactgcaaagctggttcgaaccgccaaaacctggcaccacaaagcctgagaagaagacaccgccgccgttgccgccttcctgccaggcgctactggatgagcatgtactctgatggacaatttttatgatctgtttatggtctccccgctgctgctggtggtgctgttttttgtcgccgtactggcaggatttatcgattctatcgccggaggcggagggctgctcactatccctgcgctgatggccgccgggatgtcgccggcaaacgcgttggcgaccaataaattacaggcgtgcggcggctccctctcgt

SEQ ID NO: 23 - protein, 1195 aa, Artificial Sequence
  ClyA-Toxin A repeat
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAVAAAGSVSGPFGLSISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVGPTYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIY

SEQ ID NO: 24 - DNA, 6784 bp, Artificial Sequence
  ClyA-Toxin B repeat fusion construct in ssaV and under the control of an ssaG promoter
ctgaactttcctcaacacgatatgccgttgagttttcactttctcgtcatttcaacgcgttactgaaatggttacgtaatggtgaagataaaagaggtagcgatgaatattaaaattaatgagataaaaatgacgccccctacagcatttacccctggcctggttatagaggaacaagaggttatttcgccttcaatgttagctctccatgagttacaggaaacggcgggggcagcgctctatgagacgatggaagaaataggaatggcgctgagtggtaaactgcgcgaaagtaataaattcactgatgctgagaaactggagcgcaggcagcaggctttgctgcgtttgataaaacaaatacaggaggataatggggcagcgttgcgtccgcttaccgaagagaatagtgatcctgatttacagaatgcgtatcaaattatcgctcttgcaatggcgcttactgccggcgggttgtcaaaaaagaaaaaacgcgatttgcaatcgcaactggatacgcttacagcggaggagggatgggaacttgccgtttttagtttactggaacttggcgaagtggataccgctacgctgtcctcgctgaagcgttttatgcaacaggcgatagacaacgatgaaatgcccttatcgcagtggttcagacgcgtggcagactggccggatcgcggtgaacgggtccgtattttgctaagagcaatagcctttgaacttagcatatgcatcgaaccctcggagcaaagtcgtttggccgcagcattagtacgcttgcgtcgtttgttgttattccttggccttgaaaaagagtgccagcgtgaggagtggatttgccagttgccgcctaatacattactgccgctactactcgatatcatttgtgagcgctggcttttcagcgattggttgcttgatagacttaccgctatagtttcttcatcgaagatgttcaatcggttactccaacaacttgatgcgcagtttatgctgatacccgataactgttttaacgacgaagatcaacgtgaacaaattctcgaaacgcttcgtgaagtaaagataaatcaggttttattctgatacctggctttcaatatttaggtaaattggctttctggctcatcatgaggcgtcaggatggattgggatctcattactgaacgtaatattcagctttttattcaattagcaggattagctgaacggcctttagcaaccaatatgttctggcggcaaggacaatatgaaacctgtctaaactatcataatggtcgtattcacttatgtcagatactcaagcaaaccttcttagacgaagaactgctttttaaagcgttggctaactggaaacccgcagcgttccagggtattcctcaacgattatttttgttgcgcgatgggcttgcaatgagttgttctccacctctttccagctccgccgagctctggttacgattacatcatcgacaaataaaatttctggagtcgcaatgcgttcatggttaggtgagggaatcagggcgcaacagtggctcagtgtatgcgcgggtcgtcaggatatggtcctagctcgagattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagccatatgaccagcattttcgccgaacagactgtggaagtggtgaagtcggcaatcgaaaccgcggacggcgctctggatctgtataacaaatatctggaccaggtaatcccctggaaaaccttcgatgaaacgatcaaagaactttcgaggtttaagcaggaatattcgcaggaagcctcagtcctcgtcggcgatatcaaagtgctgctcatggattctcaggataagtatttcgaagcaacgcagacggtctatgaatggtgtggggtggtcacacagttactttccgcatacatccttctgttcgatgaatacaacgaaaaaaaggcatccgcgcagaaagatatcttaatcaggattcttgatgacggtgttaagaaactgaacgaagctcagaaatcgctgcttacaagctcccagtcgttcaacaatgcgtcaggtaaactgttagcgcttgactcacagttgacaaatgatttctctgaaaagagcagttatttccagtcccaggtggatagaataagaaaagaggcatacgcggtggcagccgctggttcggtgtccgggccattcggtctgtcgatttcttatagcattgcggctggtgttatcgagggaaagctgattccggagcttaataaccgacttaagaccgtgcagaacttctttacttcactcagcgcgacagtcaagcaggccaacaaggatatcgacgccgccaaactcaagctggccacagaaattgctgcaatcggtgagataaagacagagacagaaacgacccgcttctatgtggactatgatgaccttatgttgagtctccttaaaggagccgccaaaaagatgataaacacgtgcaacgagtatcaacaaaggcatggaaaaaagacattatttgaagttccagacgttcccgggaagttttatatcaacaacttcggcatgatggtgtctggcttgatctacatcaacgatagcctctattatttcaagccgcccgttaataacttaatcacaggcttcgtgacagtaggtgatgacaaatactattttaatccgatcaatggaggcgcagcaagtattggtgaaacgataatcgacgacaagaactattattttaaccaatcaggagtgctgcaaactggtgtgttttccaccgaggacggctttaagtacttcgcccccgcgaacaccctggacgaaaaccttgagggtgaagccattgacttcactggtaaacttattatcgacgaaaacatctactattttgatgataactacagaggcgcagtggagtggaaagagctggacggggaaatgcattacttttccccagagacaggtaaagctttcaaaggtctgaatcagattggggattacaaatattacttcaactctgacggtgtcatgcagaagggatttgtgtcaatcaacgataataagcactactttgatgactcaggagtaatgaaggtgggctacacggagattgacggaaaacatttctatttcgccgaaaatggtgaaatgcagattggcgttttcaataccgaggatggcttcaagtattttgctcatcacaatgaggatctgggaaacgaagaaggcgaggaaatttcctactcgggcatactgaattttaacaataaaatatattatttcgacgacagttttacggcggttgttgggtggaaggatttagaagatggtagtaaatactacttcgatgaggacacggccgaagcctatatcggtttgtcgctgattaatgatggacagtactattttaatgacgacggcattatgcaagttgggttcgtgaccattaacgacaaagtgttttatttttcagactcaggaattatcgagagcggggttcaaaacattgatgataattatttttacatagacgataatgggatcgttcagatcggggtgttcgacacatctgacggttacaaatattttgctcccgcaaatacggtgaacgacaacatttacgggcaggcagtggaatattcgggtttggttagagttggcgaggatgtctactattttggcgagacatacacgattgaaacggggtggatttacgatatggagaacgaaagcgataaatattactttaacccagaaacaaagaaggcctgcaaaggtatcaatttaatcgatgatatcaaatactatttcgacgaaaagggtatcatgcgtactgggctgatcagctttgagaacaataattactatttcaatgaaaatggggaaatgcaatttggatatattaatatagaagataagatgttttatttcggggaggatggtgtgatgcagatcggcgttttcaacaccccggacgggtttaaatatttcgcacatcagaatacactggatgagaacttcgagggtgagtctattaactacaccgggtggctggacttagacgagaaacgctactatttcacagacgagtacattgcagctactggttcggtcatcattgatggcgaggaatattatttcgacccggataccgcccagttagtgatctccgagtaatctagactagcctaggctagtctagacttatacaagtggtagaaagtattgaccttagcgaagaggagttggcggacaatgaagaatgaattgatgcaacgtctgaggctgaaatatccgccccccgatggttattgtcgatggggccgaattcaagatgtcagcgcaacgttgttaaatgcgtggttgcctggggtatttatgggagagttgtgctgtataaagcctggagaagaacttgctgaagtcgtggggattaatggcagcaaagctttgctatctccttttacgagtactatcgggcttcactgcgggcagcaagtgatggccttaaggcgacgccatcaggttcccgtgggcgaagcgttattagggcgagtcattgatggttttggtcgtccccttgatggctgcgaactgcccgacgtctgctggaaagactatgatgcaatgcctcctcccgcaatggttcgacagcctatcactcaaccattaatgacggggattcgcgctattgatagcgttgcgacctgtggtgaagggcaacgagtgggtattttttctgctcctggcgtggggaaaagcacgcttctggcgatgctgtgtaatgcgccagacgcagactgcaatgttctggtgttaattggtgaacgtggacgagaagtccgcgagttcatcgattttacactgtctgaagagacccgaaaacgttgtgtcattgttgtcgcaacctctgacagacccgccttagagcgcgtgagggcgctgtttgtggccaccacgatagcagaattttttcgcgataatggaaaacgagtcgtcttgcttgccgactcactgacgcgttatgccagggccgcacgggaaatcgctctggccgccggagagaccgcagtttctggagaatatccgccaggcgtatttagtgcattgccacgacttttagaacgtacaggaatgggggaaaaaggcagtattaccgcattttatacggtcctggtggaaggcgatgatatgaatgagccgttggcggatgaagtccgttcactgcttgacggacatattgtgctatcccggcggcttgcagagagggggcattatcctgccattgacgtgttggcaacgctcagccgcgtttttccagtcgttaccagccatgagcatcgtcaactggcggctatattgcgacggcgcctggcgctttaccaggaggttgaactgttaatacgcattggggaataccagcgaggagttgatactgataccgataaagccattgatacctatccggatatttgcacatttttgcgacaaagtaaggatgaagtatgcggacccgagctactcatagaaaaattacatcaaatactcaccgagtgatcatggaaactttgctggagataatcgcgcggcgtgaaaagcaattacgcagcaaacttaccgtgcttgatcagcagcaacaggcgattattactgaacagcagatttgccagacgcgcgctttagcagtgactaccagactgaaagaattaatgggctggcaaggtacgttatcttgtcatttattgttggataagaaacaacaaatggccggactattcactcaggcgcagagctttttgacgcaacggcagcagttagagaatcagtatcagcagcttgtctccaggcgaagcgaattacagaagaattttaatgcgcttatgaaaaagaaagaaaaaattactatggtattaagcgatgcgtattaccaaagttgagggaagtcttgggttgccatgccagtcttatcaggatgataacgaggcggaggcggaacgtatggactttgaacaactcatgcaccaggcattacccattggtgagaataatcctcctgcagcattgaataagaacgtggttttcacgcaacgttatcgtgttagtggcggttatcttgacggtgtagagtgtgaagtctgtgagtcaggagggctaatccagttaagaatcaatgtccctcatcatgaaatttaccgttcgatgaaagcgctaaagcagtggctggagtctcagttgctgcatatggggtatataatttccctggagatattctatgttaagaatagcgaatgaagagcgtccgtgggtggagatacttccaacacaaggcgctaccattggtgagctgaca

SEQ ID NO: 25 - protein, 850 aa, Artificial Sequence; identical to SEQ ID NO: 15
  ClyA-Toxin B repeat fusion

(19) United States
(12) Patent Application Publication
(10) Pub. No.: US 2014/0044749 A1
(43) Pub. Date: Feb. 13, 2014

(54) VACCINES AGAINST CLOSTRIDIUM DIFFICILE AND METHODS OF USE

(71) Applicant: PROKARIUM LTD., Keele (GB)

(72) Inventors: Jonathan Lewis TELFER, Berkshire (GB); Lisa Caproni, Warfield (GB)

(73) Assignee: PROKARIUM LTD., Keele (GB)

(21) Appl. No.: 13/964,578

(22) Filed: Aug. 12, 2013

Related U.S. Application Data
(63) Continuation of application No. 13/057,897, filed on Oct. 3, 2011, now abandoned.
(60) Provisional application No. 61/086,673, filed on Aug. 6, 2008.

(51) Int. Cl. A61K 39/08

(52) U.S. Cl. CPC A61K 39/08
USPC 424/200.1; 435/252.3

(57) ABSTRACT

Attenuated microorganisms expressing Clostridium difficile antigen(s), and methods of using the same for vaccination of patients are disclosed The invention provides an attenuated microorganism expressing an immunogenic portion of a C difficile Toxin A C-terminal repeat region and/or a C difficile Toxin B C-terminal repeat region The microorganism is an attenuated Salmonella comprising an integrated gene expression cassette that directs the expression of the immunogenic peptide from an in vivo inducible promoter.

### RELATED INVENTIONS

This application claims priority to U.S. provisional patent application 61/086,673, filed Aug. 6, 2008, which is herein incorporated by reference in its entirety.

### FIELD OF THE INVENTION

The present invention relates to live bacterial vectors expressing Clostridium difficile antigens for vaccination against C. difficile infection, and methods of vaccination using the same.

### ACCOMPANYING SEQUENCE LISTING

The contents of the text file submitted electronically herewith are incorporated herein by reference in their entirety: A computer readable format copy of the Sequence Listing (filename: EMER_{—}001_{—}01WO SeqList_ST25.txt, date recorded: Aug. 6, 2009, file size 183 kilobytes).

### BACKGROUND

Clostridium difficile is a major cause of nosocomial diarrhea in industrialized countries. Although many cases respond to available therapy; infection can increase morbidity, prolong hospitalization, and produce life-threatening colitis. There are also major problems with infection recurrence after the initial episode.

The pathogenesis of C. difficile-associated diarrhea (CDAD) is mediated by the actions of two large protein exotoxins, toxin A and toxin B, which induce mucosal injury and inflammation of the colon.

Protective vaccination against a gut pathogen, such as C. difficile, sufficient to block the action of the associated toxins, may require the production of secretory immunoglobulin A antibodies at the mucosal site. Such antibodies may inhibit toxin from binding to brush border membranes in the colonic mucosa. To induce production of secretary Immunoglobulin A, a vaccine antigen must be properly presented to the gut-associated lymphoid tissue. Systemic immunity may also be important for protective vaccination (Aboudola, Infect. Immun. 71(3):1608-1610 (2003)), and also requires that the vaccine antigen be properly presented. For example, while the C. difficile toxins are immunogenic in both animals and humans using various immunization routes, successful vaccines have not been generated. For instance, parenteral immunization with the C. difficile toxins generates a systemic anti-toxin response which is only partially protective upon intact C. difficile challenge (Lyerly et al., Curr. Microbiol. 21:29-32 (1990)). Further, immunization of hamsters with toxin A repeats provides protection from toxin A challenge, but provides only partial protection in the animal model from subsequent challenge with intact C. difficile.

Accordingly, a vaccine for inducing protective immunity in humans against the gut pathogen C. difficile must present the vaccine antigen to the host immune system in a manner that stimulates effective immune response(s), which likely include mucosal and systemic humoral responses.

### SUMMARY OF THE INVENTION

The present invention provides attenuated microorganisms expressing Clostridium difficile antigen(s), and methods of using the same for vaccination of patients. The invention further provides recombinant C. difficile antigens and encoding polynucleotides useful for inducing immune responses against C. difficile toxin. The invention thereby provides vaccines, methods, and antigens suitable for inducing an effective immune response, e.g., including a mucosal immune response, against C. difficile infection and/or C. difficile toxin.

In one aspect, the invention provides an attenuated microorganism expressing an immunogenic peptide that comprises an immunogenic portion of a C. difficile Toxin A C-terminal repeat region and/or a C. difficile Toxin B C-terminal repeat region. The microorganism is capable of presenting the C. difficile antigen(s) to the host immune system in a manner that generates an effective immune response. In certain embodiments, the attenuated microorganism is an attenuated Salmonella comprising an integrated gene expression cassette that directs the expression of the immunogenic peptide from an in vivo inducible promoter, such as the Salmonella ssaG promoter (ssaGp), ssrA promoter (ssrAp), or sseA promoter (sseAp), for example. The immunogenic peptide may be secreted from the microorganism via a secretion signal or tag, such as ClyA or a non-hemolytic derivative thereof.

The attenuated Salmonella capable of expressing one or more C. difficile immunogenic peptides may comprise one or more gene deletions or inactivated genes. For instance, the attenuated Salmonella may comprise at least one gene deletion or inactivated gene in the Salmonella Pathogenicity Island 2 (SPI2 region). In one embodiment, the attenuated Salmonella comprises a deletion or inactivation of a ssa gene such as ssaV or ssaJ. In one embodiment, the attenuated Salmonella comprises a deletion or inactivation of at least one SPI2 gene (e.g., ssaV) and at least one gene outside of the SPI2 region, for instance, an auxotrophic gene such as aroC. In one embodiment, the gene expression cassette comprising a nucleic acid encoding the C. difficile immunogenic peptide or peptides under the control of an in vivo inducible promoter is inserted in the chromosome of the attenuated Salmonella at one or more gene deletion sites. For instance, the invention includes an attenuated Salmonella enterica serovar comprising deletion mutations in a gene of the SPI2 region and a second gene outside of the SPI2 region, wherein an gene expression cassette comprising a nucleic acid encoding a C. difficile toxin A C-terminal repeat peptide and/or toxin B C-terminal repeat peptide under the control of an in vivo inducible promoter is chromosomally inserted in the SPI2 gene deletion site and/or second gene deletion site.

In a second aspect, the invention provides a method for vaccinating a subject against a C. difficile infection or C. difficile-related condition by administering the attenuated microorganism of the invention, or composition comprising the same, to a subject. For example, the microorganism may be orally administered to a subject, such as a subject at risk of acquiring a C. difficile infection, or a subject having a C. difficile infection, including a subject having a recurrent infection. The method induces an effective immune response in the subject, which may include a mucosal immune response against C. difficile toxin. In one aspect of the invention, an attenuated microorganism of the invention is administered to a subject to induce an immune response.

In other aspects, the invention provides recombinant antigens and polynucleotides encoding the same. The recombinant antigens of the invention comprise immunogenic portions of C. difficile toxin A and/or toxin B C-terminal repeat region(s), and may be designed for expression on the surface of a bacterial vector and/or secretion from a bacterial vector, for example, by recombinant fusion with a ClyA secretion tag or a non-hemolytic derivative of ClyA. In one embodiment, the recombinant antigens and/or polynucleotides of the invention are isolated and/or purified. In another embodiment, the recombinant antigens of the invention are contained within a bacterial outer membrane vesicle, for instance, a Salmonella outer membrane vesicle. The invention includes an isolated and/or purified Salmonella outer membrane vesicle comprising the recombinant antigen of the invention. The recombinant antigens of the invention are useful for inducing an effective immune response, such as a mucosal immune response, against C. difficile toxin in a human patient.

In another embodiment of the invention, the secretion tag is an E. coli CS3 signal sequence as disclosed in U.S. provisional application 61/107,113, filed Oct. 21, 2008, which is herein incorporated by reference in its entirety.

The invention also includes methods of vaccinating a subject against a C. difficile infection or C. difficile-related condition by administering the recombinant antigens and/or polynucleotides, or composition comprising the same, to the subject. In one aspect of the invention, a recombinant antigen and/or polynucleotide, or composition comprising the same, is administered to a subject to induce an immue response.

### DESCRIPTION OF THE FIGURES

FIG. 1 shows the structure, diagrammatically, of C. difficile toxin A and toxin B. Toxin A is slightly larger than toxin B, with the toxins having molecular weights of 308 kDa and 270 kDa respectively. The two toxins have approximately 66% similarity at the amino acid level. The toxins both have an amino-terminal enzymatic domain, a putative translocation domain, and a repetitive carboxy-terminal binding domain. The amino acid sequence of the carboxy-terminal binding domain of toxins A and B is repetitive, containing long and short repeats forming solenoid folds, which are common to carbohydrate-binding proteins.

FIG. 2 depicts an ssaG antigen operon, in which an ssaG promoter controls the transcription of a gene encoding two fusions: a first fusion between the ClyA secretion tag and toxin A repeats, and a second fusion between the ClyA secretion tag and toxin B repeats.

FIG. 3 depicts plasmid pCVD aro toxAB, for creating an exemplary attenuated Salmonella in accordance with the invention. pCVD aro toxAB is a suicide vector carrying the operon shown in FIG. 2, with the flanking regions of the aroC deletion site of S. typhi ZH9. pCVD aro toxAB is designed to direct the integration of the ssaG operon to the aroC gene deletion site of S. typhi ZH9.

FIG. 4A shows a diagram including restriction map of the transcriptional fusion of FIG. 2 in aroC. FIG. 4B shows the nucleotide sequence of the transcriptional fusion (SEQ ID NO: 17) with the ssaG promoter region highlighted. Both FIGS. 4A and 4B depict the nucleic acid sequence after integration into the Salmonella genome. FIG. 4C shows the amino acid sequences of the encoded ClyA-Toxin A repeat fusion (Fusion A, SEQ ID NO: 18) and the ClyA-Toxin B repeat fusion (Fusion B, SEQ ID NO: 19).

FIG. 5A shows a diagram including restriction map of a translational fusion of ClyA-Toxin A repeats-Toxin B repeats in aroC and under the control of an ssaG promoter. FIG. 5B shows the nucleotide sequence of the translational fusion (SEQ ID NO: 20) with the ssaG promoter region highlighted. Both FIGS. 5A and 5B depict the nucleic acid sequence after integration into the Salmonella genome. FIG. 5C shows the amino acid sequences of the encoded fusion (SEQ ID NO: 21).

FIG. 6A shows a diagram of a ClyA-Toxin A repeat fusion construct in aroC and under the control of an ssaG promoter. FIG. 6B shows the nucleotide sequence of the fusion (SEQ ID NO: 22) with the ssaG promoter highlighted. Both FIGS. 6A and 6B depict the nucleic acid sequence after integration into the Salmonella genome. FIG. 6C depicts the amino acid sequence of the encoded fusion (SEQ ID NO: 23).

FIG. 7A shows a diagram with restriction map of a ClyA-Toxin B repeat fusion construct in ssaV and under the control of an ssaG promoter. FIG. 7B provides the nucleotide sequence of the ClyA-Toxin B repeat fusion construct (SEQ ID NO: 24) with the ssaG promoter region highlighted. Both FIGS. 7A and 7B depict the nucleic acid sequence after integration into the Salmonella genome. The amino acid sequence of the encoded fusion is shown in FIG. 7C (SEQ ID NO: 25).

FIG. 8 shows nucleotide and amino acid sequences for a ClyA-toxin A repeat fusion (SEQ ID NO: 12, SEQ ID NO: 13) (A) and a ClyA-toxin B repeat fusion (SEQ ID NO: 14, SEQ ID NO: 15) (B), both with linkers and codon-optimized for expression in Salmonella.

FIG. 9A shows relative mRNA levels for C. difficile toxin A terminal repetitive domain (CRD) for strains LC219 (FAFB), ZS121 (FAB) and LC5117 (FA/FB). FIG. 9B shows relative mRNA levels for C. difficile toxin B terminal repetitive domain (CRD) for strains LC219 (FAFB), ZS121 (FAB) and LC5117 (FA/FB).

### DETAILED DESCRIPTION OF THE INVENTION

### General Description

The invention provides live attenuated bacterial vaccines, recombinant antigens, recombinant polynucleotides, vaccine compositions, and methods of preventing and treating C. difficile infection and related conditions based on immunogenic portions of the C. difficile exotoxins A and/or B. The vaccine compositions of the present invention are suitable for inducing an effective immune response, e.g., including a mucosal immune response, against C. difficile infection and/or C. difficile toxin in a patient.

In one aspect, the invention provides an attenuated microorganism expressing an immunogenic peptide that comprises an immunogenic portion of a C. difficile Toxin A C-terminal repeat region and/or a C. difficile Toxin B C-terminal repeat region. The microorganism is capable of presenting the C. difficile antigen(s) to the host immune system in a manner that generates an effective immune response, e.g., when administered orally to, or to a mucosal surface of, a human or non-human animal patient.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts that term's definition in the application, the definition that appears in this application controls.

### DEFINITIONS

As used herein, the term “attenuated” refers to a bacterium that has been genetically modified so as to not cause illness in a human or animal model. The terms “attenuated” and “avirulent” are used interchangeably herein.

As used herein, the term “bacterial vaccine vector” refers to an avirulent bacterium that is used to express a heterologous antigen in a host for the purpose of eliciting a protective immune response to the heterologous antigen. The attenuated microorganisms, including attenuated Salmonella enterica serovars, provided herein are suitable bacterial vaccine vectors. Bacterial vaccine vectors and compositions comprising the same disclosed can be administered to a subject to prevent or treat a C. difficile infection or C. difficile-related condition. Bacterial vaccine vectors and compositions comprising the same can also be administered to a subject to induce an immune response. In one embodiment, the bacterial vaccine vector is the spi-VEC™ live attenuated bacterial vaccine vector (Emergent Product Development UK, UK), also known as S. typhi strain Ty2.

As used herein, the term “effective immune response” refers to an immune response that confers protective immunity. For instance, an immune response can be considered to be an “effective immune response” if it is sufficient to prevent a subject from developing a C. difficile infection after administration of a challenge dose of C. difficile or administration of C. difficile toxins. An effective immune response may comprise a humoral immune response and/or a cell mediated immune response. In one embodiment, the effective immune response refers to the ability of the vaccine of the invention to elicit the production of antibodies. An effective immune response may give rise to mucosal immunity. See, for instance, Holmgren and Czerkinsky, Nature Medicine 11:S45-S53 (2005). In one embodiment, an effective immune response gives rise to the production of anti-C. difficile peptide IgA and/or IgG antibodies.

As used herein, the term “gene expression cassette” refers to a nucleic acid construct comprising a nucleic acid encoding one or more C. difficile immunogenic peptides under the control of an inducible promoter. In one embodiment, the inducible promoter is an in vivo inducible promoter. The gene expressiong cassette may additionally comprise, for instance, one or more of a nucleic acid encoding a secretion signal and a nucleic acid encoding a peptide linker. A gene expression cassette may be contained on a plasmid or may be chromosomally integrated, for instance, at a gene mutation (e.g., deletion) site. A microorganism may be constructed to contain more than one gene expression cassette.

As used herein, the term “immunogenic peptide” refers to a portion of a C. difficile toxin capable of eliciting an immunogenic response when administered to a subject. An immunogenic peptide can be a C-terminus repeating unit of toxin A or toxin B (also known as combined repetitive oligopeptides (CROPs) or C-terminal repetitive domain (CRD)) and variants thereof capable of eliciting an immunogenic response.

The term “pharmaceutically acceptable” means approved by a regulatory agency of the Federal or a state government or listed in the U.S. Pharmacopeia or other generally recognized pharmacopeia for use in animals, and more particularly in humans.

As used herein, the term “promoter” refers to a region of DNA involved in binding RNA polymerase to initiate transcription.

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

As used herein, the term “secretion signal” refers to a peptide that causes a co-expressed immunogenic peptide to be directed to the surface of an attenuated microorganism, to be secreted from the attenuated microorganism and/or to “bleb” off the attenuated microorganism. The secretion signal may stay intact or be removed partially or entirely during the routing of the immunogenic peptide. The terms secretion signal, secretion tag, secretion sequence, export tag, export peptide, and export sequence are used interchangeably herein.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences may be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison windon for polypeptide sequences may be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window may comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

“Variants or variant” refers to a nucleic acid or polypeptide differing from a reference nucleic acid or polypeptide, but retaining essential properties thereof. Generally, variants are overall closely similar, and, in many regions, identical to the reference nucleic acid or polypeptide. For instance, a variant may exhibit at least about 70%, at least about 80%, at least about 90%, at least about 95%, at least about 96%, at least about 97%, at least about 98% or at least about 99% sequence identity compared to the active portion or full length reference nucleic acid or polypeptide. In one embodiment, “variant” refers to a C. difficile toxin A or toxin B fragment such as the C-terminal repeating region of toxin A or toxin B that differs in sequence from the corresponding native C. difficile toxin A or toxin B but retaining at least one functional and/or therapeutic property thereof as described elsewhere herein or otherwise known in the art. In another embodiment, the variant is a nucleic acid sequence that has been codon-optimized for expression in a particular host. For instance, the invention includes a codon-optimized nucleic acid sequence that encodes a C. difficile toxin A or toxin B C-terminal repeating region or fragment thereof.

C. difficile Immunogenic Peptide

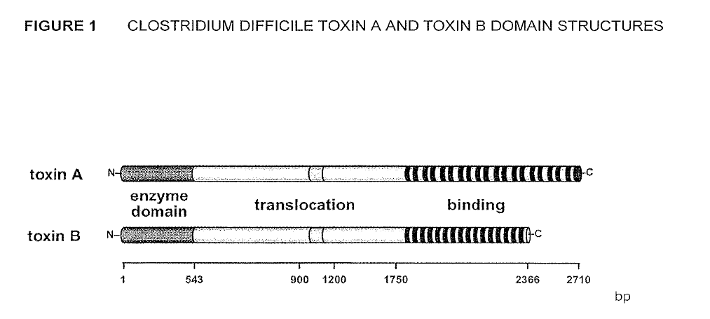

The pathogenesis of C. difficile-associated diarrhoea (CDAD) is mediated by the actions of two large protein exotoxins, toxin A and toxin B, which induce mucosal injury and inflammation of the colon. Toxin A is slightly larger than toxin B, the toxins having molecular weights of about 308 kDa and about 270 kDa respectively. While toxin A may be the primary mediator of tissue damage within the intestine, toxin B may act after the initial toxin A-mediated damage thus exacerbating the mucosal tissue damage. The toxins consist of an amino-terminal enzymatic domain, a putative translocation domain and the repetitive carboxy-terminal binding domain. See FIG. 1. The two toxins are homologous (having approximately 66% similarity at the amino acid level), and are thought to have arisen through a gene duplication event.

A feature of both toxin A and B is the repetitive nature of the amino acid sequences located at the carboxyl terminus of the protein. Specifically, long and short repeats form solenoid folds, the structure of which was recently solved for toxin A (Ho et al., PNAS 102 p 18373-18378, 2005). This sequence/structure is common to certain carbohydrate-binding proteins. Antiserum raised against the repeat region was found to neutralize the cytotoxic activity of toxin A (Lyerly et al., Curr. Microbiol. 21 p 29-33, 1990). In addition, studies with a synthetic decapeptide corresponding to a hydrophilic sequence conserved within the repeats showed that even this short sequence possessed a receptor-binding capability, and that antiserum raised against the peptide could partially inhibit the binding and cytotoxic activity of whole toxin A (Wren et al., Infect. Immun. 59 p 3151-3155, 1991).

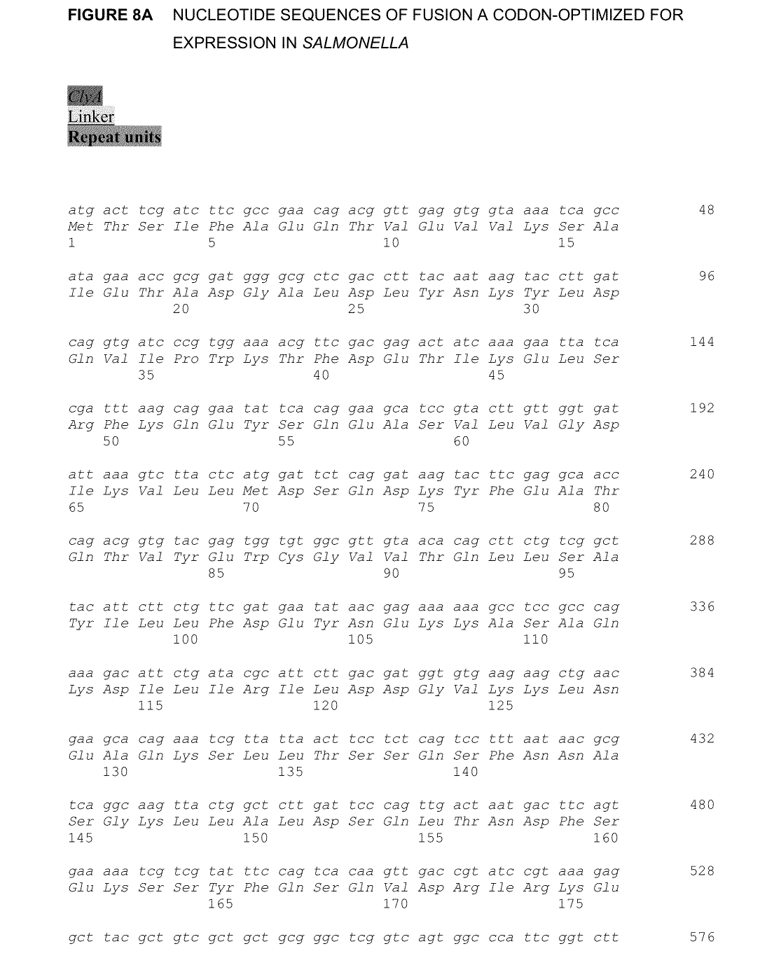

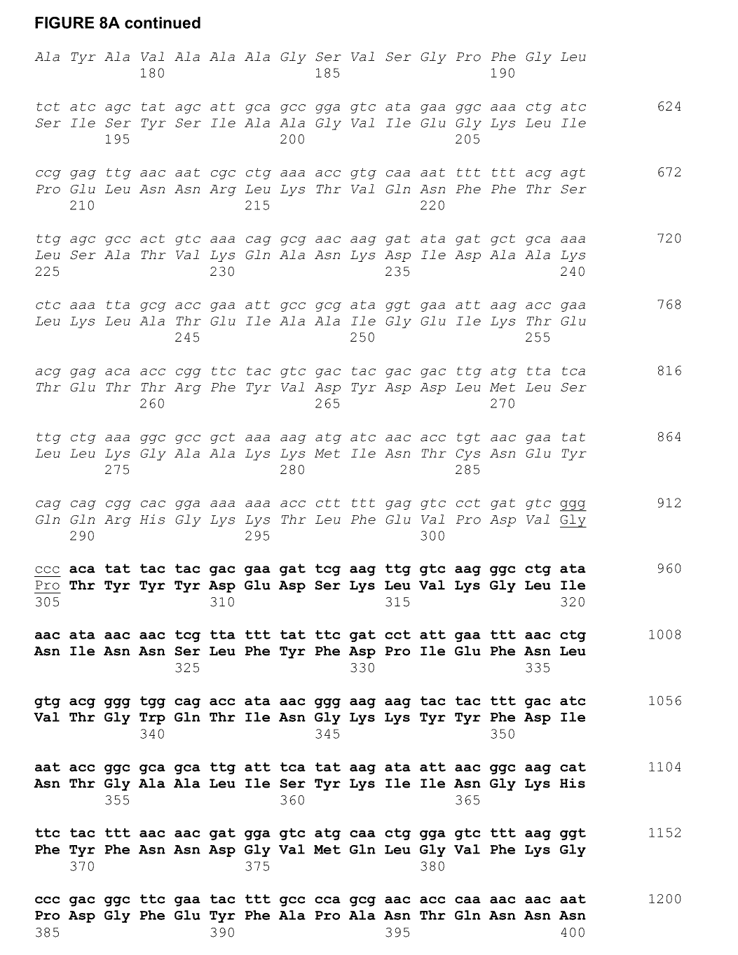

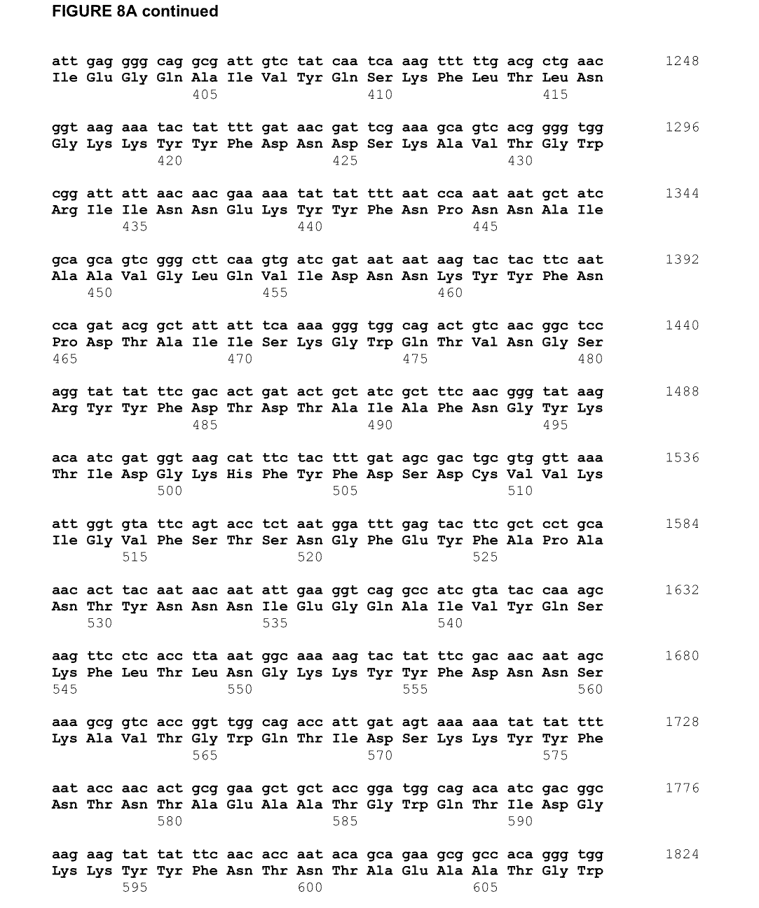

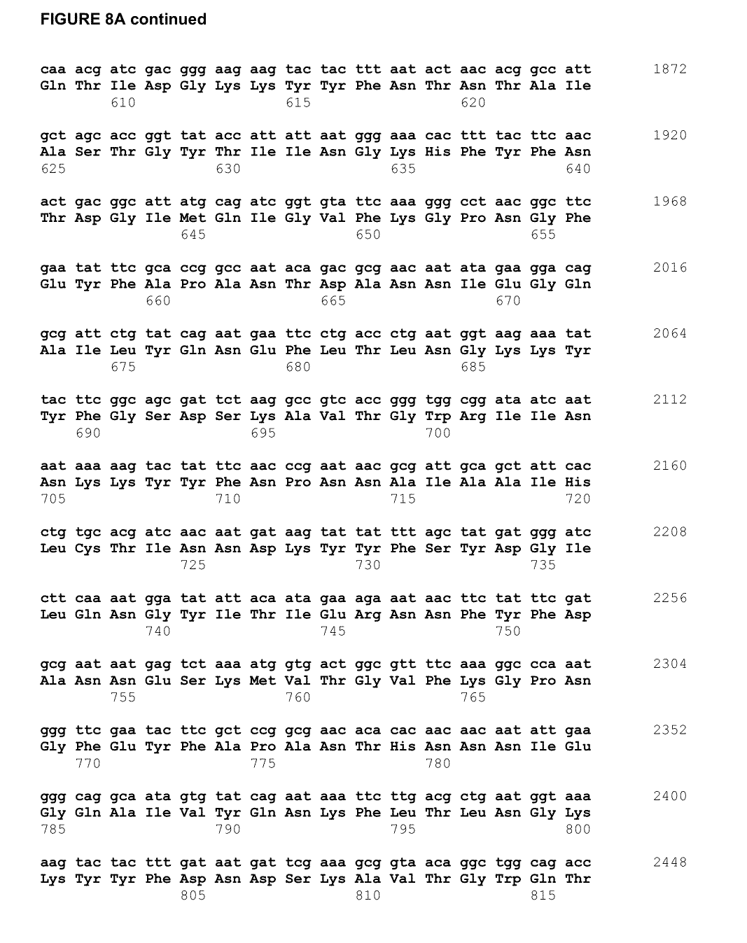

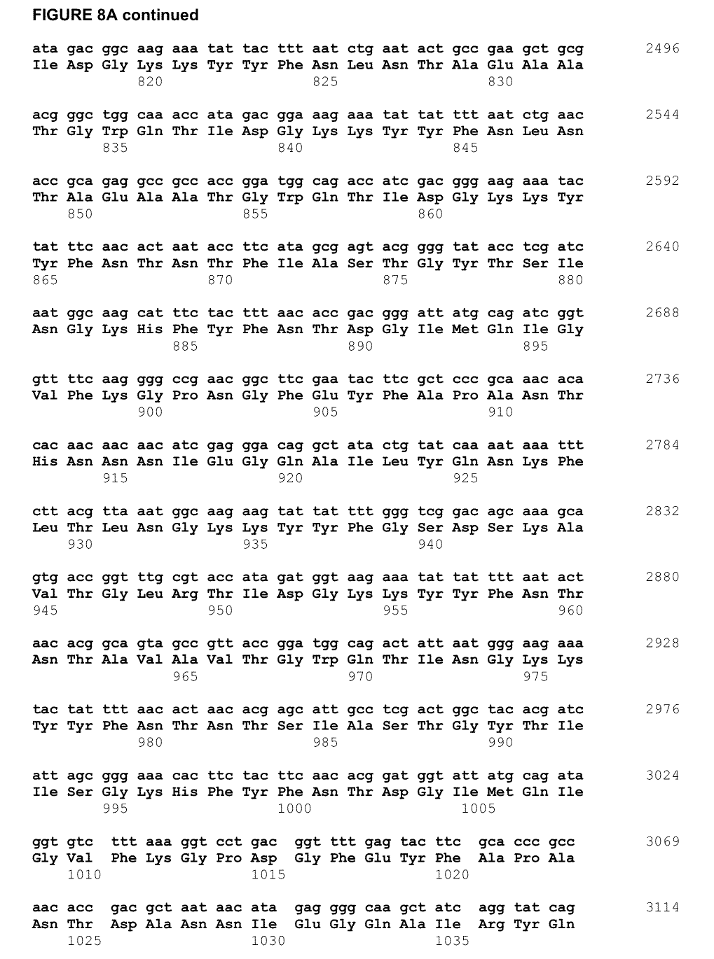

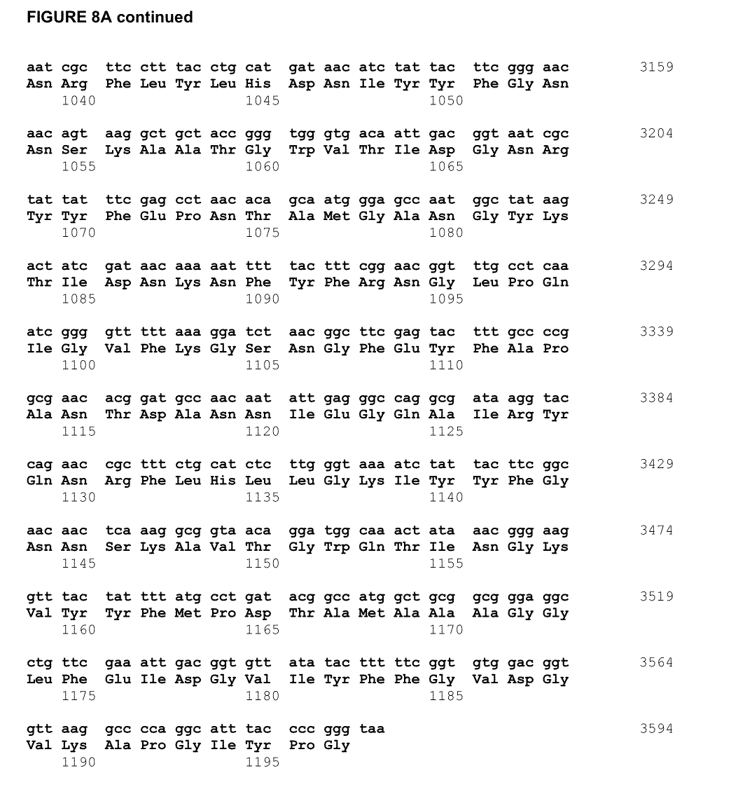

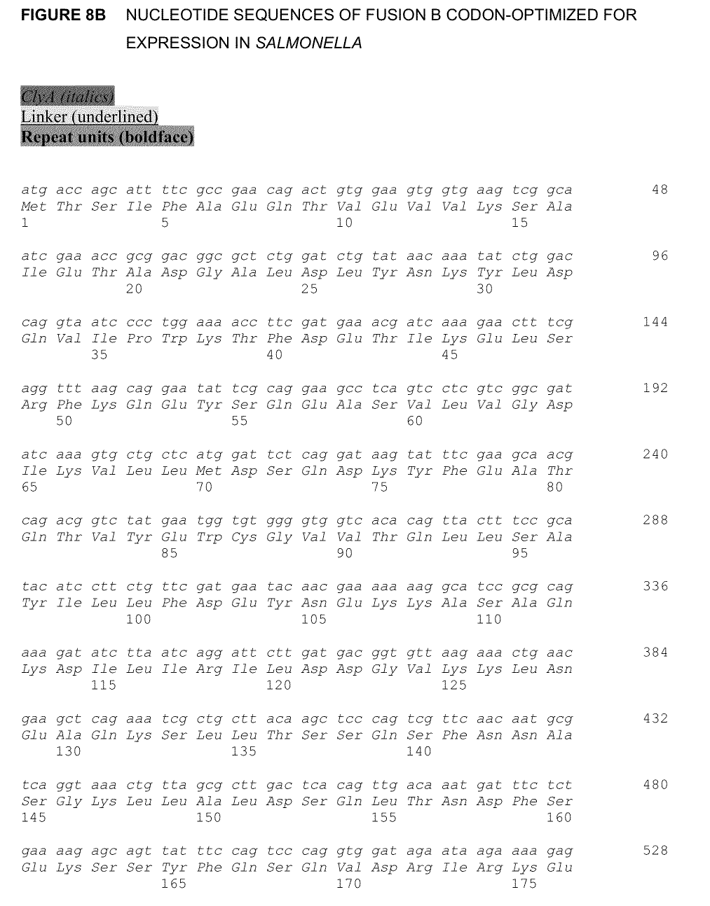

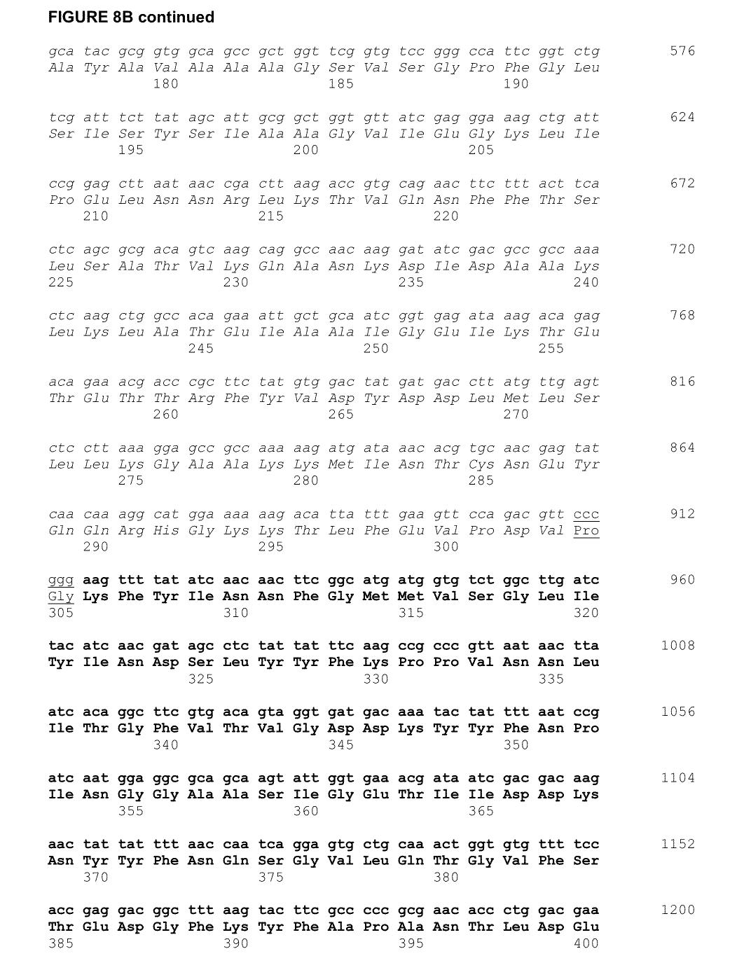

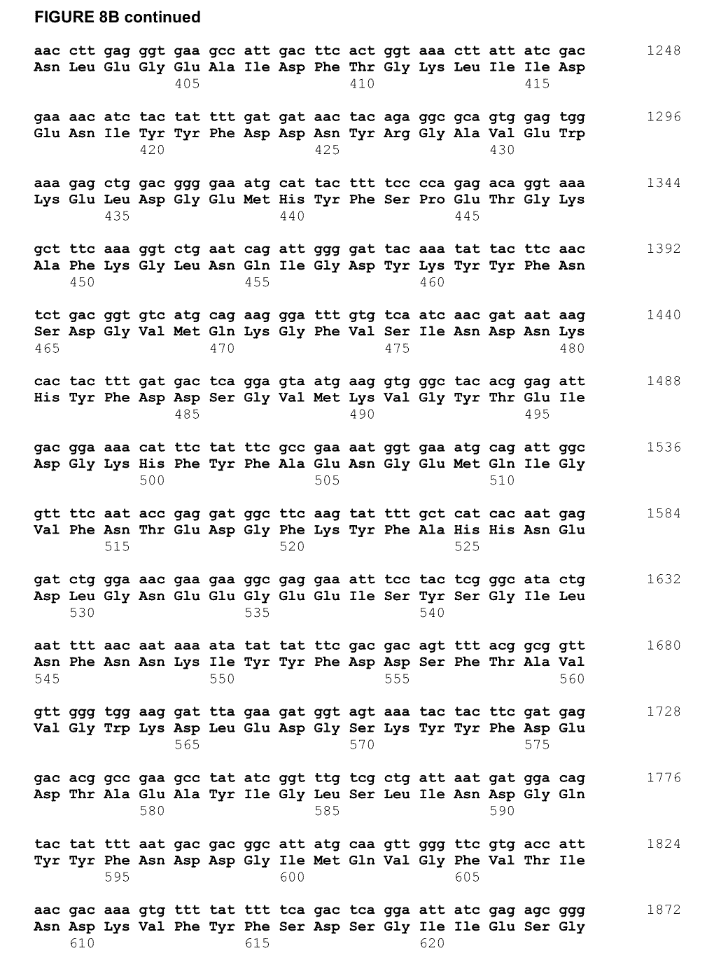

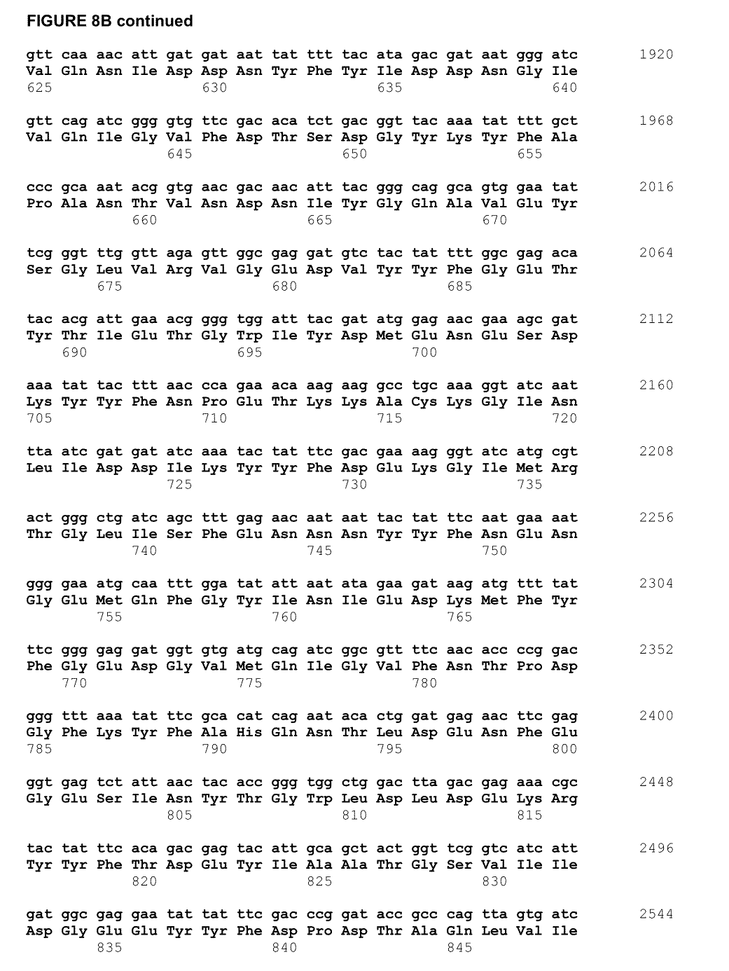

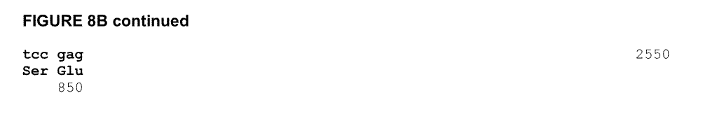

The present invention provides vaccines, and particularly, live attenuated bacterial vectors, expressing immunogenic portions of the toxin A and/or toxin B C-terminal repeat region(s). Generally, the toxin A C-terminal repeat region and/or the Toxin B C-terminal repeat region each comprise at least about 3, 4, 5, 6, 7, 8, 9, 10, 11, 12, 13, 14, 15, 16. 17, 18, 19, 20, 21, 22, 23, 24 or 25 repeat units. For example, the immunogenic portion of the toxin A C-terminal repeat region may comprise at least about 20 or 25 repeat units, such as about 28 repeat units. The immunogenic portion of the toxin B C-terminal repeat region may comprise at least about 15 repeats, such as about 17 repeats. Exemplary amino acid sequences and encoding nucleotide sequences for exemplary immunogenic toxin A and toxin B repeat regions are presented in FIG. 8. Such sequences may be modified in accordance with the invention, so long as the desired immunogenicity is maintained, that is, so long as the modified toxins are capable of inducing the production of antibodies that are cross-reactive with the wild-type C. difficile exotoxins. Such modified sequences may include amino acid sequences having at least about 50%, 75%, 80%, 85%, 90%, 95%, 96%, 97%, 98% or 99% or greater sequence identity with corresponding portions of toxin A (SEQ ID NO: 2) and/or B (SEQ ID NO: 4).

### Secretion Signals

The attenuated microorganism and/or immunogenic peptide may be constructed so as to express on the cell surface and/or secrete one or more immunogenic peptides, each immunogenic peptide comprising portions of one or more C. difficile antigens, for instance, C-terminal repeat regions of toxin A and/or toxin B. A strong antibody response to the antigen, e.g., systemic and/or mucosal, may be elicited by expression of the immunogenic peptide on the cell surface or secretion of the immunogenic peptide.

In certain embodiments, the immunogenic peptide is designed for cell surface expression or secretion by a bacterial export system. In one embodiment, the immunogenic peptide is secreted by a ClyA export system, e.g., by engineering the expressed immunogenic peptide to include a ClyA secretion signal. ClyA and its use for secretion of proteins from host cells is described in U.S. Pat. No. 7,056,700, which is hereby incorporated by reference in its entirety. Generally, the ClyA export system expresses the immunogenic peptide in close association with membranous vesicles, which may increase the potency of the immune response. Further, the ClyA export system may secrete the immunogenic peptide, which can be sizeable, in a manner that preserves and presents the necessary epitopes for presentation to the host immune system.

Other secretion systems that may find use with the invention include other members of the HlyE family of proteins. The HlyE family consists of HlyE and its close homologs from E. coli, Shigella flexneri, S. typhi, and other bacteria. E. coli HlyE is a functionally well characterized, pore-forming, chromosomally-encoded hemolysin. It consists of 303 amino acid residues (34 kDa). HlyE forms stable, moderately cation-selective transmembrane pores with a diameter of 2.5-3.0 nm in lipid bilayers. The crystal structure of E. coli HlyE has been solved to 2.0 angstrom resolution, and visualization of the lipid-associated form of the toxin at low resolution has been achieved by electron microscopy. The structure exhibits an elaborate helical bundle about 100 angstroms long. It oligomerizes in the presence of lipid to form transmembrane pores.

This haemolysin family of proteins (of which ClyA is a member, SEQ ID NO: 5) typically cause haemolysis in eukaryotic target cells. Thus, the secretion signal may be modified in some embodiments so as to be non-hemolytic or have reduced hemolytic activity. Such modifications may include modifications at one or more, or all of, positions 180, 185, 187, and 193 of ClyA (SEQ ID NO: 6). In certain embodiments, the ClyA secretion signal has one or more or all the following modifications: G180V, V185S or I185S, A187S, and I193S. However, alternative modifications to the wild-type sequence may be made, so long as the ClyA secretion signal is substantially non-hemolytic. Such modifications may be guided by the structure of the protein, reported in Wallace et al., E. coli Hemolysin E (HlyE, ClyA, SheA): X-Ray Crystal Structure of the Toxin and Observation of Membrane Pores by Electron Microscopy, Cell 100:265-276 (2000), which is hereby incorporated by reference in its entirety. For example, modifications may include modification of outward-facing hydrophobic amino acids in the head domain to amino acids having hydrophilic side chains.

ClyA sequences that may be used and/or modified to export the immunogenic peptide include S. typhi clyA (available under GENBANK Accession No. AJ313034) (SEQ ID NO: 7); Salmonella paratyphi clyA (available under GENBANK Accession No. AJ313033) (SEQ ID NO: 8); Shigella flexneri truncated HlyE (hlyE), the complete coding sequence available under GENBANK Accession No. AF200955 (SEQ ID NO: 9); and the Escherichia coli hlyE, available under GENBANK Accession No. AJ001829 (SEQ ID NO: 10).

Thus, the immunogenic peptide may be secreted from the microorganism via a secretion signal, such as the ClyA secretion signal, or non-hemolytic derivative thereof. The immunogenic peptide may be engineered as a recombinant fusion of a ClyA secretion tag, and a C. difficile Toxin A and/or Toxin B C-terminal repeat region. In some embodiments, the recombinant fusion comprises a fusion of ClyA and the Toxin B C-terminal repeat region, or comprises a fusion of ClyA and the Toxin A C-terminal repeat region, or comprises a fusion of ClyA and both the Toxin A and Toxin B C-terminal repeat regions. In certain embodiments, the ClyA secretion signal is separated from the toxin domains by a linker sequence to, for example, maintain the functional independence of the secretion signal.

Other secretion sequences may be used to secrete the immunogenic peptide from the bacterial host cell, including, but not limited to secretion sequences involved in the Sec-dependent (general secretory apparatus) and Tat-dependent (twin-arginine translocation) export systems. For instance, a leader sequence from S. typhi sufl can be used (msfsrrqflgasgialcagaiplranaagqqqplpyppllesuggplfm (SEQ ID NO: 11) to export the immunogenic peptide. Additional export system sequences comprising the consensus sequence s/strrxfl plus a hydrophobic domain can be used to export the immunogenic peptide from the bacterial host cell.

It is envisioned that signal sequences and secretion sequences known in the art can be used to export the immunogenic peptide out of the live, attenuated microorganism and into the host, including the host gastrointestinal tract. Such sequences can be derived, for instance, from viruses, eukaryotic organisms and heterologous prokaryotic organisms. See, for instance, U.S. Pat. Nos. 5,037,743; 5,143,830 and 6,025,197 and US Patent Application 20040029281, for disclosure of additional signal sequences and secretion sequences.

In one embodiment of the invention, the secretion sequence is cleaved from the exported immunogenic peptide. In other embodiment of the invention, the bacterial secretion sequence is not cleaved from the exported immunogenic peptide, but, rather, remains fused so as to create a secretion tag and immunogenic peptide fusion protein. For instance, the invention includes a fusion protein comprising a ClyA secretion sequence fused to one or more immunogenic peptides. In other embodiment of the invention, the bacterial secretion sequence maintains the conformation of the immunogenic peptide.

In another embodiment of the invention, the secretion sequence causes the exported immunogenic peptide to “bleb off” the bacterial cell, i.e., a bacterial outer-membrane vesicle containing the immunogenic peptide is released from the bacterial host cell. See Wai et al., Vesicle-Mediated Export and Assembly of Pore-Forming Oligomers of the Enterobacterial ClyA Cytotoxin, Cell 115:25-35 (2003), which is hereby incorporated by reference in its entirety. The invention includes avirulent bacterial vesicles comprising one or more immunogenic peptides of the invention. In one embodiment, avirulent bacterial vesicles comprise a secretion sequence fused to the one or more immunogenic peptides and, optionally, one or more linker peptides. For instance, the invention includes a S. enterica vesicle comprising a ClyA export sequence fused to a C. difficile C-terminus repeat region of toxin A and/or toxin B.

In another embodiment of the invention, the secretion signal is an enterotoxigenic E. coli surface antigen 3 (CS3) peptide as disclosed in U.S. provisional application 61/107,113, filed Oct. 21, 2008, which is herein incorporated by reference in its entirety. In enterotoxigenic E. coli, full length CS3 protein forms fimbriae, which extend from the bacterial cell surface and facilitate the attachment of the bacteria to the intestinal epithelium. Fusion proteins comprising CS3 or fragments thereof can be targeted to the outer surface of host cells, where they are effectively presented to the immune system and induce an immune response. An example of a nucleic acid sequence that encodes a CS3 secretion signal is atgttaaaaataaaatacttattaataggtctttcactgtcagctatgagttcatactcactagct (SEQ ID NO: 26). An example of a CS3 secretion signal is MLKIKYLLIGLSLSAMSSYSLA (SEQ ID NO: 27).

### Peptide Linker

In one embodiment, a peptide linker is used to separate the secretion signal from an immunogenic peptide. In another embodiment, a peptide linker is used to separate two immunogenic peptides, for instance, a C. difficile C-terminal repeating region of toxin A and a C-terminal repeating region of toxin B. Accordingly, the present invention includes an attenuated Salmonella bacterium capable of expressing (a) a fusion protein comprising a secretion signal+linker+C. difficile immunogenic peptide, (b) a fusion protein comprising a secretion signal+linker+C. difficile immunogenic peptide+linker+C. difficile immunogenic peptide, and/or (c) a fusion protein comprising a C. difficile immunogenic peptide+linker+C. difficile immunogenic peptide.

In another embodiment, the invention includes a fusion protein comprising (a) a secretion signal+linker+C. difficile immunogenic peptide, (b) a fusion protein comprising a secretion signal+linker+C. difficile immunogenic peptide+linker+C. difficile immunogenic peptide, and/or (c) a fusion protein comprising a C. difficile immunogenic peptide+linker+C. difficile immunogenic peptide.

In yet another embodiment, the invention includes a vaccine comprising (a) a secretion signal+linker+C. difficile immunogenic peptide, (b) a fusion protein comprising a secretion signal+linker+C. difficile immunogenic peptide+linker+C. difficile immunogenic peptide, and/or (c) a fusion protein comprising a C. difficile immunogenic peptide+linker+C. difficile immunogenic peptide. The vaccine can be a live, attenuated bacterial vector vaccine or a polypeptide vaccine. In one embodiment, the polypeptide is contained within a bacterial membrane that is lacking genomic DNA.

Without wishing to be bound by a particular theory, in some instances, it is believed that the peptide linker allows the C. difficile immunogenic peptide to maintain correct folding. The linker peptide may also assist with the effective presentation of the C. difficile immunogenic peptide outside of the Salmonella cell, in particular by providing spatial separation from the secretion tag and/or other C. difficile immunogenic peptide. For example, the peptide linker may allow for rotation of the C. difficile immunogenic peptide amino acid sequence(s) and secretion signal relative to each other.

In one embodiment of the invention, the live, attenuated Salmonella comprises a nucleic acid sequence encoding a peptide linker of about 1, 2, 3, 4, 5, 6, 7, 8, 9, 10, 11, 12, 13, 14 or 15 amino acids in length.

In one embodiment, the linker comprises or consists essentially of glycine, proline, serine, alanine, threonine, and/or asparagine amino acid residues. In one embodiment of the invention, the peptide linker comprises or consists essentially of glycine and/or proline amino acids. For instance, in one embodiment, the peptide linker comprises the amino acid sequence GC. In another embodiment, the peptide linker comprises the amino acid sequence CG.

In one embodiment, the peptide linker comprises or consists essentially of glycine and/or serine amino acids. In one embodiment, the peptide linker comprises or consists essentially of proline amino acids. In one embodiment, the peptide linker comprises or consists essentially of glycine amino acids.

### Live, Attenuated Bacterial Vaccine Vector

In one embodiment of the invention, the immunogenic portions of the C. difficile exotoxins, as described above, are presented to the host immune system via a live, attenuated bacterial vaccine vector, such as an attenuated gram negative bacterial vaccine vector. Exemplary microbial vectors include Vibrio cholerae, Shigella spp. and Salmonella spp., as well as others described in U.S. Pat. No. 5,877,159, which is hereby incorporated by reference in its entirety. In various embodiments, the bacterial vector is an attenuated Salmonella enterica serovar, for instance, S. enterica serovar Typhi, S. enterica serovar Typhimurium, S. enterica serovar Paratyphi, S. enterica serovar Enteritidis, S. enterica serovar Choleraesuis, S. enterica serovar Gallinarum, S. enterica serovar Dublin, S. enterica serovar Hadar, S. enterica serovar Infantis and S. enterica serovar Pullorum.

Generally, the microorganism carries one or more gene deletions or inactivations, rendering the microorganism attenuated. In certain embodiments, the microorganism is attenuated by deletion of all or a portion of a gene(s) associated with pathogenicity. Further, such deletions may be affected by replacement of the one or more genes associated with pathogenesis, with a gene expression cassette expressing the immunogenic portions of one or more of C. difficile toxin A and/or toxin B. Alternatively, the gene(s) may be inactivated, for example, by mutation in an upstream regulatory region or upstream gene so as to disrupt expression of the pathogenesis-associated gene, thereby leading to attenuation. For instance, a gene may be inactivated by an insertional mutation.

In certain embodiments, the attenuated microorganism may be an attenuated gram negative bacterium as described in U.S. Pat. Nos. 6,342,215; 6,756,042 and 6,936,425, each of which is hereby incorporated by reference in its entirety. For example, the microorganism may be an attenuated Salmonella spp. (e.g., S. enterica Typhi or S. enterica Typhimurium) comprising a first deletion or inactivation in a gene located within the Salmonella pathogenicity island 2 (SPI2). The present invention includes an attenuated Salmonella spp. with more than one deleted or inactivated SPI2 genes.

SPI2 is one of more than two pathogenicity islands located on the Salmonella chromosome. SPI2 comprises several genes that encode a type III secretion system involved in transporting virulence-associated proteins, including SPI2 so-called effector proteins, outside of the Salmonella bacteria and potentially directly into target host cells such as macrophages. SPI2 apparatus genes encode the secretion apparatus of the type III system. SPI2 is essential for the pathogenesis and virulence of Salmonella in the mouse. S. typhimurium SPI2 mutants are highly attenuated in mice challenged by the oral, intravenous and intraperitoneal routes of administration.

Infection of macrophages by Salmonella activates the SPI2 virulence locus, which allows Salmonella to establish a replicative vacuole inside macrophages, referred to as the Salmonella-containing vacuole (SCV). SPI2-dependent activities are responsible for SCV maturation along the endosomal pathway to prevent bacterial degradation in phagolysosomes, for interfering with trafficking of NADPH oxidase-containing vesicles to the SCV, and remodeling of host cell microfilaments and microtubule networks. See, for instance, Vazquez-Torres et al., Science 287:1655-1658 (2000), Meresse et al., Cell Microbiol. 3:567-577 (2001) and Guignot et al., J. Cell Sci. 117:1033-1045 (2004), each of which is herein incorporated by reference in its entirety. Salmonella SPI2 mutants are attenuated in cultured macrophages (see, for instance, Deiwick et al., J. Bacteriol. 180(18):4775-4780 (1998) and Klein and Jones, Infect. Immun. 69(2):737-743 (2001), each of which is herein incorporated by reference in its entirety). Specifically, Salmonella enterica SPI2 mutants generally have a reduced ability to invade macrophages as well as survive and replicate within macrophages.

The deleted or inactivated SPI2 gene may be, for instance, an apparatus gene (ssa), effector gene (sse), chaperone gene (ssc) or regulatory gene (ssr). In certain embodiments, the attenuated Salmonella microorganism is attenuated via a deletion or inactivation of a SPI2 apparatus gene, such as those described in Hensel et al., Molecular Microbiology 24(1):155-167 (1997) and U.S. Pat. No. 6,936,425, each of which is herein incorporated by reference in its entirety. In certain embodiments, the attenuated Salmonella carries a deletion or inactivation of at least one gene associated with pathogenesis selected from ssaV, ssaJ, ssaU, ssaK, ssaL, ssaM, ssaO, ssaP, ssaQ, ssaR, ssaS, ssaT, ssaU, ssaD, ssaE, ssaG, ssaI, ssaC (spiA) and ssaH. For example, the attenuated Salmonella may carry a deletion and/or inactivation of the ssaV gene. Alternatively, or in addition, the microorganism carries a mutation within an intergenic region of ssaJ and ssaK. The attenuated Salmonella may of course carry additional deletions or inactivations of the foregoing genes, such as two, three, or four genes.

In certain embodiments, the attenuated Salmonella microorganism comprises a deletion or inactivation of a SPI2 effector gene. For instance, in certain embodiments, the attenuated Salmonella comprises a deletion or inactivation of at least one gene selected from sseA, sseB, sseC, sseD, sseE, sseF, sseG, sseL and spiC (ssaB). SseB is necessary is necessary to prevent NADPH oxidase localization and oxyradical formation at the phagosomal membrane of macrophages. SseD is involved in NADPH oxidase assembly. SpiC is an effector protein that is translocated into Salmonella-infected macrophages and interferes with normal membrane trafficking, including phagosome-lysosome fusion. See, for instance, Hensel et al., Mol. Microbiol., 30:163-174 (1998); Uchiya et al., EMBO J., 18:3924-3933 (1999); and Klein and Jones, Infect. Immun., 69(2):737-743 (2001), each of which is herein incorporated by reference in its entirety. The attenuated Salmonella may of course carry additional deletions or inactivations of the foregoing genes, such as two, three, or four genes.

In certain embodiments, the attenuated Salmonella microorganism comprises a deleted or inactivated ssr gene. For instance, in certain embodiments, the attenuated Salmonella comprises a deletion or inactivation of at least one gene selected from ssrA (spiR) and ssrB. ssrA encodes a membrane-bound sensor kinase (SsrA), and ssrB encodes a cognate response regulator (SsrB). SsrB is responsible for activating transcription of the SPI2 type III secretion system and effector substrates located outside of SPI2. See, for instance, Coombes et al., Infect. Immun., 75(2):574-580 (2007), which is herein incorporated by reference in its entirety.

In other embodiments, the attenuated Salmonella comprises an inactivated SPI2 gene encoding a chaperone (ssc). For instance, in certain embodiments, the attenuated Salmonella comprises a deletion or inactivation of one or more from sscA and sscB. See, for instance, U.S. Pat. No. 6,936,425, which is herein incorporated by reference in its entirety.

Further, the attenuated Salmonella may comprise one or more additional attenuating mutations outside of the SPI2 region. For instance, the attenuated Salmonella may carry an “auxotrophic mutation,” for example, a mutation that is essential to a biosynthetic pathway. The biosynthetic pathway is generally one present in the microorganism, but not present in mammals, such that the mutants cannot depend on metabolites present in the treated patient to circumvent the effect of the mutation. For instance, the present invention includes an attenuated Salmonella with a deleted or inactivated gene necessary for the biosynthesis of aromatic amino acids. Exemplary genes for the auxotrophic mutation in Salmonella, include an aro gene e.g., aroA, aroC, aroD and aroE. In one embodiment, the invention comprises a Salmonella SPI2 mutant comprising an attenuating mutation in the aroA gene. In addition to aro gene mutations, the present invention includes an attenuated Salmonella with the deletion or inactivation of a purA, purE, asd, cya and/or crp gene.

In another embodiment, the attenuated Salmonella SPI2 mutant also comprises at least one additional deletion or inactivation of a gene in the Salmonella Pathogenicity Island I region (SPI1). In yet another embodiment, the Salmonella SPI2 mutant comprises at least one additional deletion or inactivation of a gene outside of the SPI2 region which reduces the ability of Salmonella to invade a host cell and/or survive within macrophages. For instance, the second mutation may be the deletion or inactivation of a rec or sod gene. In yet another embodiment, the Salmonella spp. comprises the deletion or inactivation of a transcriptional regulator that regulates the expression of one or more virulence genes (including, for instance, genes necessary for surviving and replicating within macrophages). For instance, the Salmonella SPI2 mutant may further comprise the deletion or inactivation of one or more genes selected from the group consisting of phoP, phoQ, rpoS and slyA.

In certain embodiments, the attenuated microorganism is a Salmonella microorganism having attenuating mutations in a SPI2 gene (e.g., ssa, sse, ssr or ssc gene) and an auxotrophic gene located outside of the SPI2 region. In one embodiment, the attenuated microorganism is a Salmonella enterica serovar comprising a deletion or inactivation of an ssa, sse and/or ssr gene and an auxotrophic gene. For instance, the invention includes an attenuated Salmonella enterica serovar with deletion or inactivating mutations in the ssaV and aroC genes (for example, a microorganism derived from Salmonella enterica Typhi ZH9, as described in U.S. Pat. No. 6,756,042, which description is hereby incorporated by reference) or ssaJ and aroC genes.

Where the attenuated microorganism is a Salmonella bacterium, the polynucleotides segments encoding portions of the C. difficile toxins may be codon-optimized for expression in the Salmonella enterica serovar. For instance, the C. difficile toxin genes are large and have a G+C content of 28.2% compared to 51% for S. Typhi. Expression of the antigens may therefore be improved if the G+C content and codon usage are adjusted closer to that of S. enterica Typhi. See, for instance, FIGS. 8A and 8B (SEQ ID NO: 12 and SEQ ID NO: 14) which contain codon optimized nucleic acid sequences for expression of C. difficile C-terminal repeats of toxin A and toxin B, respectively, in S. typhi. The invention also includes, for instance, nucleic acids encoding immunogenic peptides that are codon optimized for expression in S. enterica Typhimurium.

### Promoter

The immunogenic peptide comprising immunogenic portions of the C. difficile toxins A and/or B and, optionally, a fused secretion signal and/or linker peptide, may be expressed by the live, attenuated bacterial vaccine vector via an inducible or constitutive promoter.

In one embodiment, the gene expression cassette may comprise a promoter that is inducible so that the immunogenic peptide is only expressed under the particular physiological conditions. In certain embodiments, the inducible promoter is a prokaryotic inducible promoter. For instance, the inducible promoter of the invention includes a gram negative bacterium promoter, including, but not limited to, a Salmonella promoter. In certain embodiments, the inducible promoter is an in vivo inducible promoter. By “in vivo inducible promoter,” it is meant that the promoter is only induced in vivo or may be induced in vitro under conditions that mimic an in vivo environment. Generally, in vivo inducible promoters are difficult to induce in vitro and genes under control of the promoter may be expressed at a much lower rate than would occur in vivo.

In certain embodiments, the inducible promoter directs expression of an immunuogenic peptide and, optionally, a fused secretion signal and/or linker peptide, within the gastrointestinal tract of the host. In certain embodiments, the inducible promoter directs expression of the immunogenic peptide and, optionally, fused secretion signal and/or linker peptide, within the gastrointestinal tract and/or immune cells (for instance, macrophages) of the host.

In certain embodiments, the inducible promoter directs expression of an immunogenic peptide (optionally, fused to a secretion tag and/or linker peptide) under acidic conditions. For instance, in certain embodiments, the inducible promoter directs expression of an immunogenic peptide at a pH of less than or about pH 7, including, for instance, at a pH of less than or about pH 6, pH 5, pH 4, pH 3 or pH 2.

The promoter of the invention can also be induced under conditions of low phosphate concentrations. In one embodiment, the promoter is induced in the presence of low pH and low phosphate concentration such as the conditions that exist within macrophages. In certain embodiments, the promoter of the invention is induced under highly oxidative conditions such as those associated with macrophages.

The promoter of the invention can be a Salmonella SPI2 promoter. In one embodiment, the microorganism is engineered such that the SPI2 promoter that directs expression of the immunogenic peptide (optionally fused to a secretion tag and/or linker peptide) is located in a gene cassette outside of the SPI2 region or within a SPI2 region that is different from the normal location of the specified SPI2 promoter. Examples of SPI2 promoters include the ssaG promoter, ssrA promoter, sseA promoter and promoters disclosed, for instance, in U.S. Pat. No. 6,936,425.

In certain embodiments, the promoter directs the expression of the immunogenic peptide under conditions and/or locations in the host so as to induce systemic and/or mucosal immunity against the antigen, including the ssaG, ssrA, sseA, pagC, nirB and katG promoters of Salmonella. The in vivo inducible promoter may be as described in WO 02/072845, which is hereby incorporated by reference in its entirety.

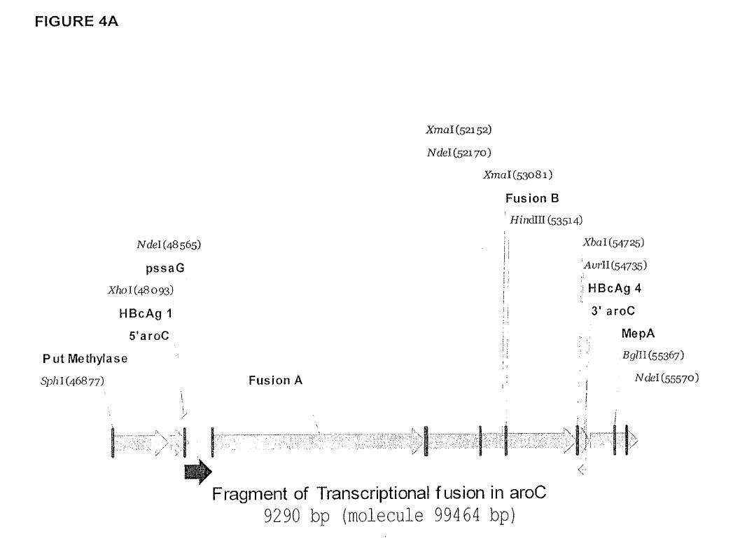

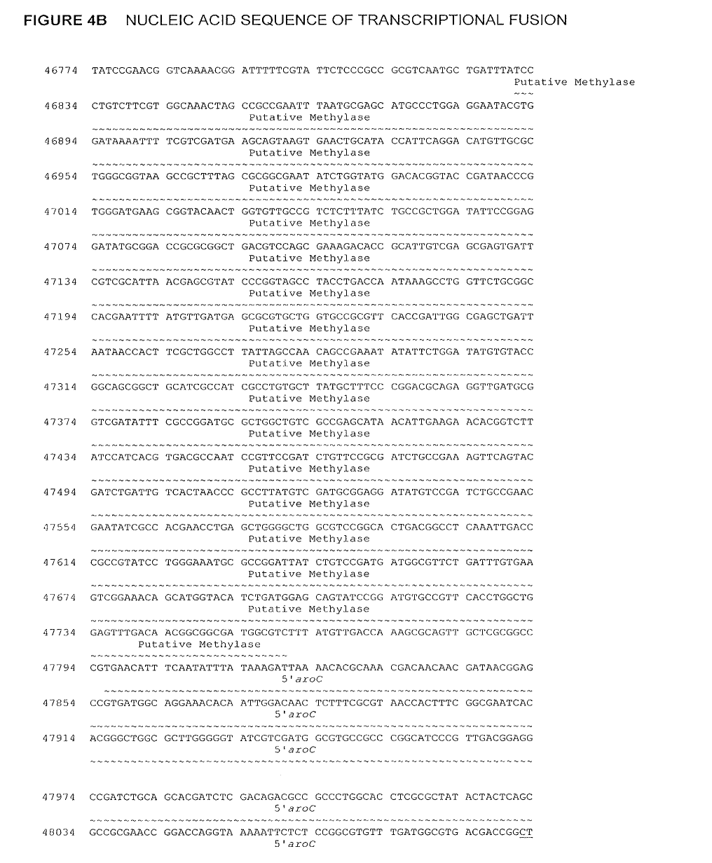

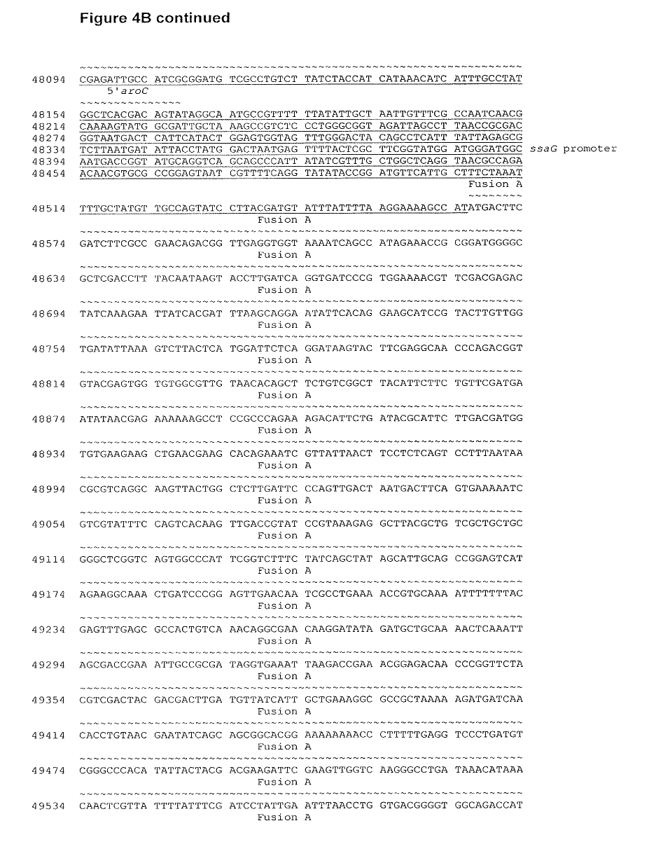

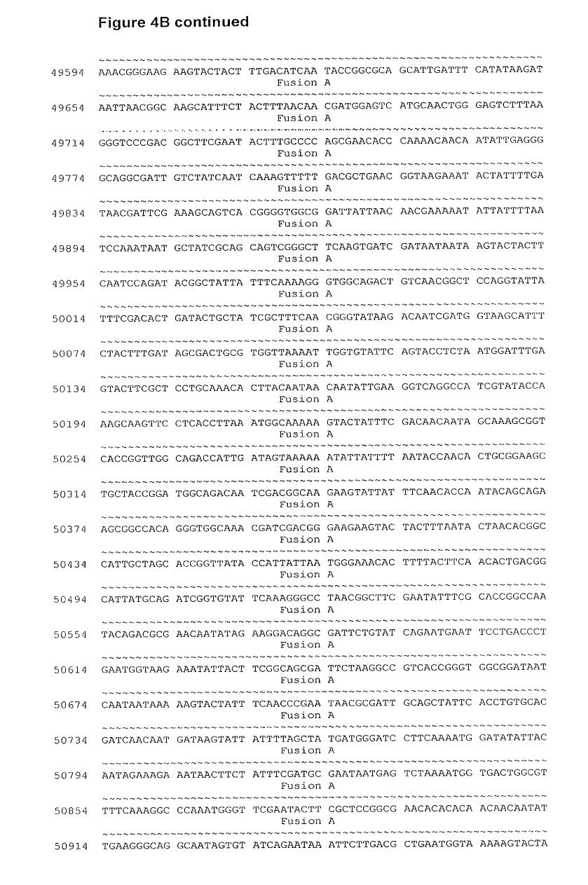

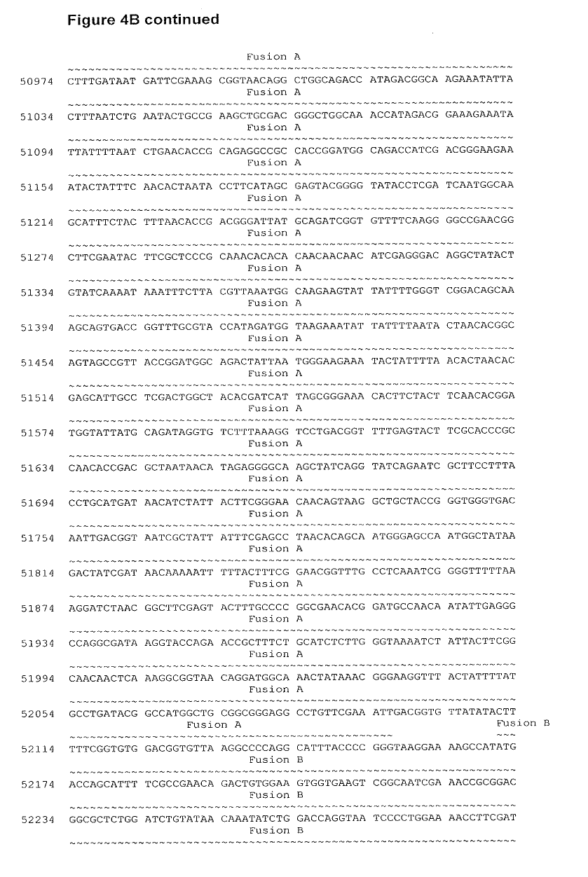

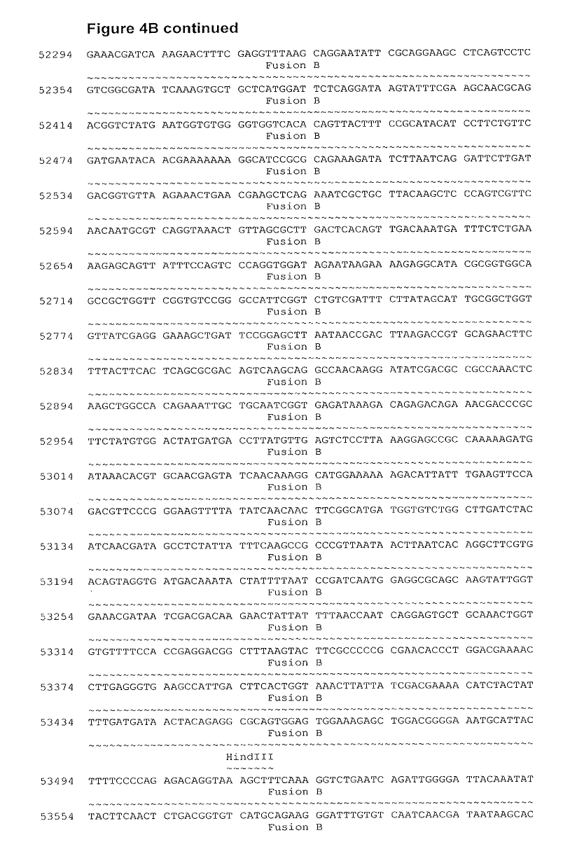

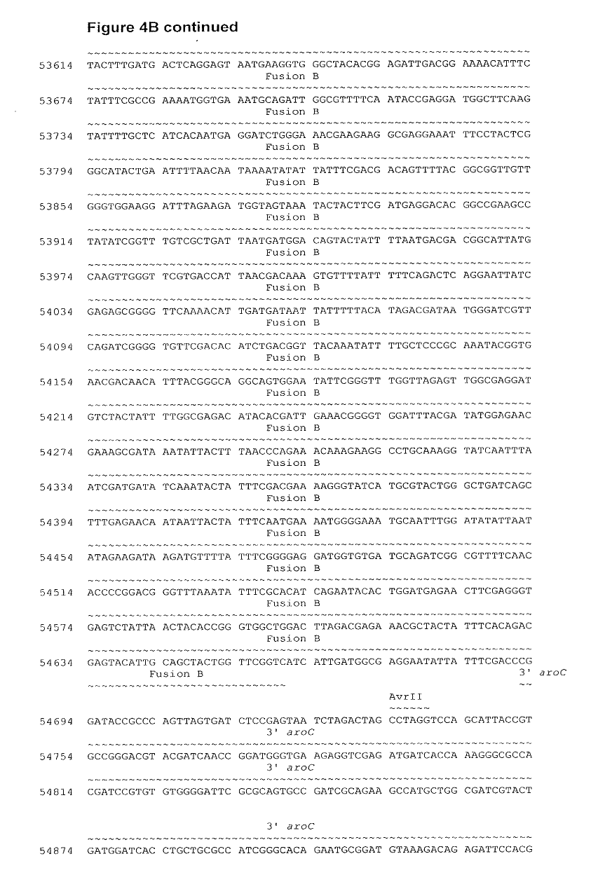

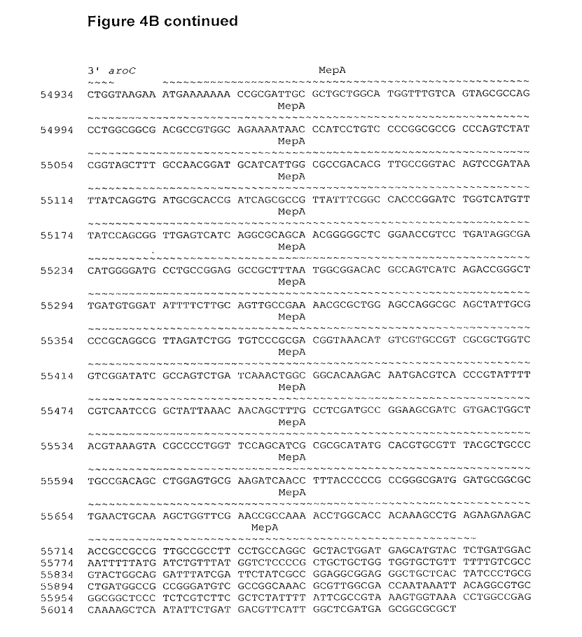

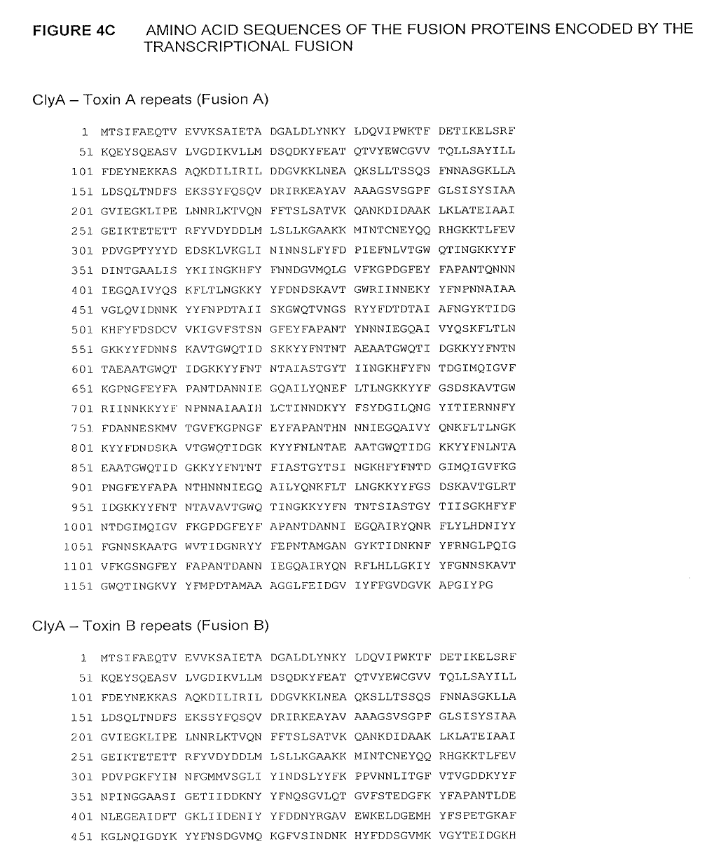

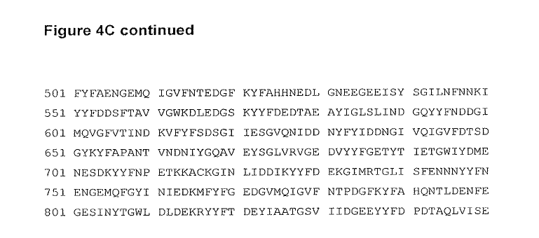

In certain embodiments, the expression of the immunogenic peptide and, optionally, fused secretion signal and/or linker peptide, by the attenuated microorganism may be controlled by a Salmonella ssaG promoter. The ssaG promoter is normally located upstream of the start codon for the ssaG gene, and may comprise the nucleotide sequence of SEQ ID NO: 16 or the sequences underlined in FIGS. 4B, 5B, 6B, and 7B. In this context, the term “ssaG promoter” includes promoters having similar or modified sequences, and similar or substantially identical promoter activity, as the wild-type ssaG promoter, and particularly with respect to its ability to induce expression in vivo. Similar or modified sequences may include nucleotide sequences with high percent sequence identity to SEQ ID NO: 16 (or those ssaG sequences highlighted in the Figures), such as nucleotide sequences having at least about 70%, 80%, 90%, 95%, 97%, 98% or 99% sequence identity to SEQ ID NO: 16 (or the ssaG promoter sequences underlined in the FIGS. 4B, 5B, 6B and 7B), as well as functional fragments, including functional fragments with high identity to corresponding functional fragments of SEQ ID NO: 16 (or the ssaG promoter sequences highlighted in the Figures). In certain embodiments, the functional ssaG promoter fragment comprises at least about 30 nucleotides, at least about 40 nucleotides, or at least about 60 nucleotides. For instance, the invention includes a promoter sequence with at least about 70%, 90%, 90%, 95%, 97%, 98% or 99% sequence identity over, for instance, at least 30 nucleotides, 40 nucleotides or 60 nucleotides.

The ssaG promoter, in some embodiments, comprises at least the sequence of about nucleotides 330 to 503 (173 bp) of SEQ ID NO: 16, or at least the sequence of about nucleotides 229 to 503 (275 bp) of SEQ ID NO: 16, or the sequence of about nucleotide 39 to 503 (465 bp) of SEQ ID NO: 16.

### Recombinant Nucleic Acid

The polynucleotide encoding the immunogenic peptide, e.g., as a recombinant fusion with a secretion signal, and under the control of an inducible promoter, may be contained on an extrachromosomal plasmid, or may be integrated into the bacterial chromosome by methods known in the art. In certain embodiments, the microorganism is an attenuated Salmonella comprising an integrated gene expression cassette that directs the expression of the immunogenic peptide from an inducible promoter. In such embodiments, the expression of the immunogenic peptide comprising the C. difficile Toxin A C-terminal repeat region and/or the C. difficile Toxin B C-terminal repeat region, is controlled by a Salmonella in vivo promoter (e.g., ssaG promoter).

In some embodiments, a polynucleotide segment encoding a fusion between a non-hemolytic ClyA export signal and a toxin A C-terminal repeat region (Fusion A), and a second polynucleotide segment encoding a fusion between a non-hemolytic ClyA export signal and the toxin B C-terminal repeat region (Fusion B), are co-transcribed from a single promoter (e.g., ssaG promoter). In these embodiments, the antigen genes will be included as a linked operon to coordinate expression and simplify construction of the vaccine strain. Alternatively, the expression of Fusion A and Fusion B are each controlled separately by independent promoters, such as two independent ssaG promoters. In still other embodiments, the immunogenic peptide comprises a recombinant fusion between the ClyA export signal, the toxin A repeat region, and the toxin B repeat region (Fusion AB), thereby providing a single translational fusion for presenting the C. difficile antigens to the host immune system.

In certain embodiments, for example, where the attenuated microorganism is derived from Salmonella enterica serovar Typhi ZH9, the toxin A C-terminal repeat region and/or the toxin B C-terminal repeat region is inserted at the aroC and/or ssaV gene deletion site. For example, a polynucleotide encoding a fusion of ClyA and the toxin A C-terminal repeat region under control of an in vivo inducible promoter may be integrated at the aroC gene deletion site; and a polynucleotide encoding a fusion of ClyA and the Toxin B C-terminal repeat region under control of an in vivo inducible promoter may be integrated at the ssaV gene deletion site. Exemplary vaccine strains in accordance with the invention are shown in Table 1.

### Recombinant Antigens

In other aspects, the invention provides recombinant antigens and polynucleotides encoding the same. The recombinant antigens of the invention comprise immunogenic portions of C. difficile toxin A and/or toxin B C-terminal repeat region(s) (as described herein), and may be designed for secretion from a bacterial vector such as Salmonella. The recombinant antigens of the invention are useful for inducing an effective immune response, such as a mucosal immune response, against C. difficile toxin in a human patient.

The recombinant antigens of the invention may, in some embodiments, comprise the toxin A C-terminal repeat region and/or the Toxin B C-terminal repeat region, where each comprise at least about 5 repeat units, or at least about 15 repeat units. For example, the immunogenic portion of the toxin A C-terminal repeat region may comprise at least about 20 or 25 repeat units, such as about 28 repeat units. The immunogenic portion of the toxin B C-terminal repeat region may comprise at least about 15 repeats, such as about 17 repeats. Exemplary amino acid sequences and encoding nucleotide sequences for exemplary immunogenic toxin A and toxin B repeat regions are presented in FIG. 8. As described, such sequences may be modified in accordance with the invention, so long as the desired immunogenicity is maintained, that is, so long as the modified toxins are capable of inducing the production of antibodies that are cross-reactive with the wild-type C. difficile exotoxins. Such modified sequences may include amino acid sequences having at least about 50%, 60%, 70%, 75%, 80%, 85%, 90%, 95%, 96%, 97%, 98% or 99% sequence identity with corresponding portions of toxin A (SEQ ID NO: 2) and/or B (SEQ ID NO: 4). For instance, the modified sequences may include amino acid sequences having at least about 70%, 75%, 80%, 85%, 90%, 95%, 96%, 97%, 98% or 99% sequence identity over at least about 10, 15, 20, 25, 30 or 35 amino acids of SEQ ID NO: 2 and/or SEQ ID NO: 4. In another embodiment of the invention, the modified sequences comprise at least about 10, 15, 20, 25, 30 or 35 contiguous amino acids of SEQ ID NO: 2 and/or SEQ ID NO: 4.

The recombinant antigens of the invention further comprise a ClyA secretion signal, as described. For example, the recombinant antigen may comprise a ClyA secretion signal fused to an immunogenic portion of a C. difficile Toxin A C-terminal repeat region, and/or an immunogenic portion of a C. difficile Toxin B C-terminal repeat region. Such recombinant antigens may further comprise a linker between the ClyA secretion signal and the Toxin A C-terminal repeat region, and/or between the ClyA secretion signal and the C. difficile Toxin B C-terminal repeat region. Exemplary recombinant antigens are shown in FIG. 8.

Alternatively, the recombinant antigen may comprise: a ClyA secretion signal, an immunogenic portion of a C difficile Toxin A C-terminal repeat region, and an immunogenic portion of a C. difficile Toxin B C-terminal repeat region. In such embodiments, the recombinant antigen provides a single fusion designed to export immunogenic portions of both toxin A and toxin B from a host microorganism, such as Salmonella. The recombinant polypeptide may further comprise a linker between the ClyA secretion signal and the Toxin A C-terminal repeat region or the C. difficile Toxin B C-terminal repeat region, to maintain the functional independence of the components.

The invention includes an isolated recombinant antigen. The recombinant antigen can be isolated by methods known in the art. An isolated recombinant antigen can purified, for instance, substantially purified. An isolated recombinant antigen can be purified by methods generally known in the art, for instance, by electrophoresis (e.g., SDS-PAGE), filtration, chromatography, centrifugation, and the like. A substantially purified recombinant antigen can be at least about 60% purified, 65% purified, 70% purified, 75% purified, 80% purified, 85% purified, 90% purified or 95% or greater purified.

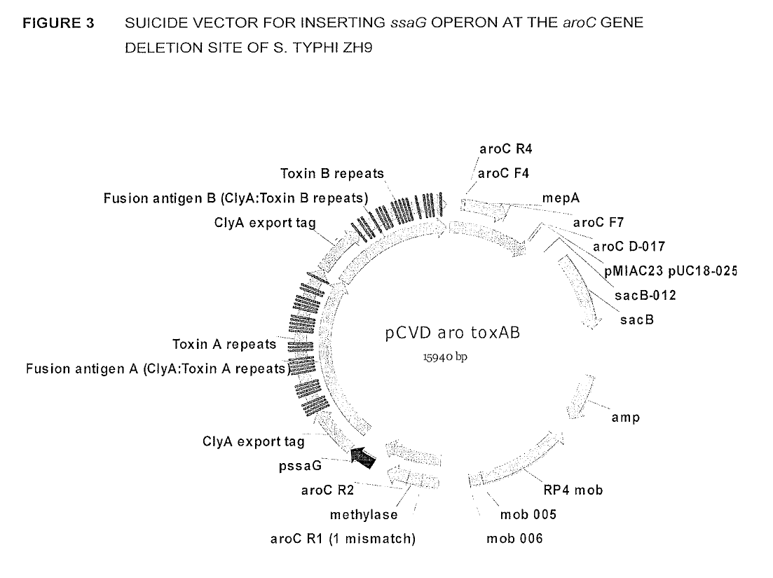

The invention further provides a polynucleotide encoding the recombinant antigens of the invention. Such recombinant antigens may be under the control of an inducible promoter as described, such as a Salmonella ssaG promoter, for example. The polynucleotide may be designed for integration at, or integrated at, an aroC and/or ssaV gene deletion site of a Salmonella host cell. In some embodiments, the polynucleotide of the invention is a suicide vector for constructing a microorganism of the invention, as exemplified in FIG. 3. The invention includes an isolated and/or purified polynucleotide. By “isolated,” it is meant that the polynucleotide is substantially free of other nucleic acids, e.g., at least about 20% pure, preferably at least about 40% pure, more preferably about 60% pure, even more preferably about 80% pure, most preferably about 90% pure, and even most preferably about 95% pure, as determined by agarose gel electrophoresis. A polynucleotide can be isolated or purified by methods generally known in the art.

### Vaccine Formulation and Administration

The microorganism may be formulated as a composition for delivery to a subject, such as for oral delivery to a human patient. In addition, the invention also includes the formulation of the recombinant antigen as a composition for delivery to a subject, such as oral delivery to a human patient. In one embodiment, the recombinant antigen may be contained within a bacterial outer membrane vesicle.

In one embodiment of the invention, the vaccine comprises one or more C. difficile immunogenic peptides or is capable of expressing one or more C. difficile immunogenic peptides in a subject. In another embodiment, the vaccine further comprises one or more immunogenic peptides from a second pathogenic organism or which is capable of expressing one or more immunogenic peptides from a second pathogenic organism. For instance, the bacterial vaccine vector of the invention can be engineered to additionally express an immunogenic peptide from a second, third or fourth enteric pathogen. In one embodiment, the second enteric pathogen is enterotoxoxigenic E. coli (ETEC) and the peptide is the ETEC heat labile toxin or heat stable toxin or variant or fragment thereof.

The composition may comprise the microorganism as described, and a pharmaceutically acceptable carrier, for instance, a pharmaceutically acceptable vehicle, excipient and/or diluent. The pharmaceutically acceptable carrier can be any solvent, solid or encapsulating material in which the vaccine can be suspended or dissolved. The pharmaceutically acceptable carrier is non-toxic to the inoculated individual and compatible with the live, attenuated microorganism.

Suitable pharmaceutical carriers are known in the art, and include, but are not limited to, liquid carriers such as saline and other non-toxic salts at or near physiological concentrations. Suitable pharmaceutical excipients include starch; amino acids, sugars (such as glucose, lactose, sucrose and trehalose), gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical vehicles, excipients and diluents are described in “Remington's Pharmaceutical Sciences” by E. W. Martin, which is hereby incorporated by reference in its entirety.

In one embodiment of the invention, the composition comprises one or more of the following carriers: disodium hydrogen phosphate, soya peptone, potassium dihydrogen phosphate, ammonium chloride, sodium chloride, magnesium sulphate, calcium chloride, sucrose, sterile saline and sterile water. In one embodiment of the invention, the composition comprises an attenuated Salmonella enterica serovar (e.g., Typhi or Typhimurium) with deleted or inactivated SPI2 (e.g., ssaV) and aroC genes and one or more gene expression cassettes comprising a nucleic acid encoding a C. difficiles toxin A and/or toxin B C-terminal repeating unit under the control of an in vivo inducible promoter (e.g., ssaG promoter) and a carrier comprising, for instance, at least one of disodium hydrogen phosphate, soya peptone, potassium dihydrogen phosphate, ammonium chloride, sodium chloride, magnesium sulphate, calcium chloride, sucrose, sodium bicarbonate and sterile water.

In certain embodiments, the compositions further comprise at least one adjuvant or other substance useful for enhancing an immune response. For instance, the invention includes a composition comprising a live, attenuated Salmonella bacterium of the invention with a CpG oligodeoxynucleotide adjuvant. Adjuvants with a CpG motif are described, for instance, in US Patent Application 20060019239, which is herein incorporated by reference in its entirety.

Other adjuvants that can be used in a vaccine composition with the attenuated microorganism of the invention, include, but are not limited to, aluminium salts (e.g., Alhydrogel) such as aluminium hydroxide, aluminum oxide and aluminium phosphate, oil-based adjuvants such as Freund's Complete Adjuvant and Freund's Incomplete Adjuvant, mycolate-based adjuvants (e.g., trehalose dimycolate), bacterial lipopolysaccharide (LPS), peptidoglycans (e.g., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), muramyldipeptides, Immune Stimulating Comlexes (the “Iscoms” as disclosed in EP 109 942, EP 180 564 and EP 231 039), saponins, DEAE-dextran, neutral oils (such as miglyol), vegetable oils (such as arachis oil), liposomes, polyols, the Ribi adjuvant system (see, for instance, GB-A-2 189 141), vitamin E, Carbopol or interleukins, particularly those that stimulate cell mediated immunity.

In certain embodiments, the compositions may comprise a carrier useful for protecting the microorganism from the stomach acid or other chemicals, such as chlorine from tap water, that may be present at the time of administration. For example, the microorganism may be administered as a suspension in a solution containing sodium bicarbonate and ascorbic acid (plus aspartame as sweetener).

Suitable formulations for oral administration include hard or soft gelatin capsules, pills, tablets, including coated tablets, sachets, elixirs, suspensions, syrups or inhalations and controlled release forms thereof. Gelatin capsules and sachets, for instance, can serve as carriers for lypholized vaccines.

The compositions of the present invention can be administered via parenteral, subcutaneous, intravenous, intramuscular, intraperitoneal, transdermal and buccal routes. Alternatively, or concurrently, administration may be noninvasive by either the oral, inhalation, nasal, or pulmonary route.

Suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

In certain embodiments, the vaccine dosage is 1.0×10⁵ to 1.0×10¹⁵ CFU/ml or cells/ml. For instance, the invention includes a vaccine with about 1.0×10⁵, 1.5×10⁵, 1.0×10⁶, 1.5×10⁶, 1.0×10⁷, 1.5×10⁷, 1.0×10⁸, 1.5×10⁸, 1.0×10⁹, 1.5×10⁹, 1.0×10¹⁰, 1.5×10¹⁰, 1.0×10¹¹, 1.5×10¹¹, 1.0×10¹², 1.5×10¹², 1.0×10¹³, 1.5×10¹³, 1.0×10¹⁴, 1.5×10¹⁴ or about 1.0×10¹⁵ CFU/ml or cells/ml. In certain embodiments, the dosage administered will be dependent upon the age, health, and weight of the recipient, kind of concurrent treatment, if any, frequency of treatment, and the nature of the effect desired.

In certain embodiments, the compositions of this invention may be co-administered along with other compounds typically prescribed for the prevention or treatment of a C. difficile infection or related condition according to generally accepted medical practice.

In a second aspect, the invention provides a method for vaccinating a subject against C. difficile by administering an attenuated microorganism of the invention, or composition comprising the same, to a patient. For example, the microorganism may be orally administered to a patient, such as a patient at risk of acquiring a C. difficile infection, or a patient having a C. difficile infection, including a patient having a recurrent infection. Accordingly, the present invention includes methods of preventing and treating a C. difficile infection comprising administering a composition comprising an attenuated microorganism of the invention.

The method of the invention induces an effective immune response in the patient, which may include a mucosal immune response against C. difficile toxin. In certain embodiments, the method of the invention may reduce the incidence of (or probability of) recurrent C. difficile infection. In other embodiments, the vaccine or composition of the invention is administered to a patient post-infection, thereby ameliorating the symptoms and/or course of the illness, as well as preventing recurrence. Symptoms of C. difficile infection and/or C. difficile-related conditions that can be prevented, reduced or ameliorated by administering the composition of the invention include, for instance, diarrhea, abdominal pain, nausea, enteritis, kidney failure, bowel perforation, toxic megacolon death and pseudomembranous colitis.

The vaccine may be administered to the patient once, or may be administered a plurality of times, such as one, two, three, four or five times.

The vaccines of the invention can also be used to prepare compositions comprising neutralizing antibodies that immunoreact with C. difficile and/or C. difficile toxin A and/or toxin B. Antisera obtained from a subject vaccinated with the vaccine of the invention can be used for the manufacture of a medicament for treating a C. difficile infection, preventing a first occurance of a C. difficile infection or preventing reoccurance of a C. difficile infection. For instance, antibodies may be isolated and substantially purified for administration to a subject at risk for developing a C. difficile infection (e.g., immunocompromised patient or elderly patient in hospital or nursing home). The antisera, or antibodies purified from the antisera, can also be used as diagnostic agents to detect C. difficile and/or C. difficile toxin A and/or toxin B.

### EXAMPLES

### Example 1

### Design of Attenuated Microorganisms

Four exemplary vaccines were produced using the S. typhi ZH9 strain (also referred to as spi-VEC vector), which contains deletion mutations in the ssaV and aroC genes (Hindle et al., Infect. Immun., 70(7):3457-3467 (2002). Also see U.S. Pat. No. 6,756,042, which is hereby incorporated by reference in its entirety. The four exemplary vaccine strains are summarized in Table 1.

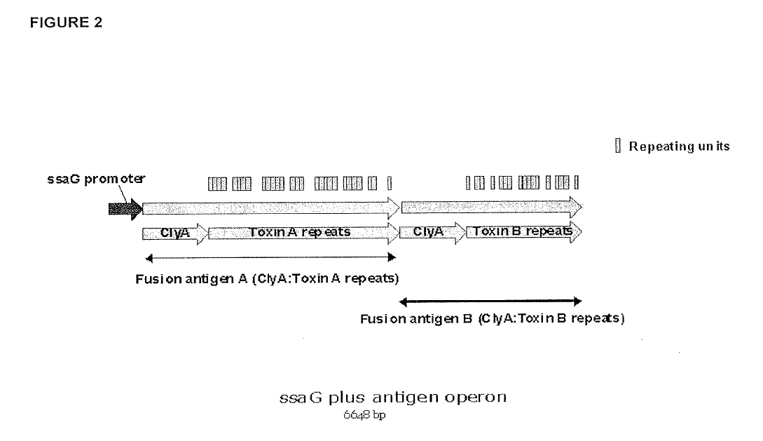

A first vaccine strain was designed to express a transcriptional fusion encoding Fusion A and Fusion B (FIG. 4C) clyA-toxin A C-terminal repeat-clyA-toxin B C-terminal repeat) under control of the ssaG promoter. The first vaccine strain contains an insertion of the operon shown diagrammatically in FIG. 2 and FIG. 4A. The nucleotide and amino acid sequences of the operon are shown in FIGS. 4B and 4C, respectively. The operon is inserted at the aroC gene deletion site of S. typhi ZH9 strain.

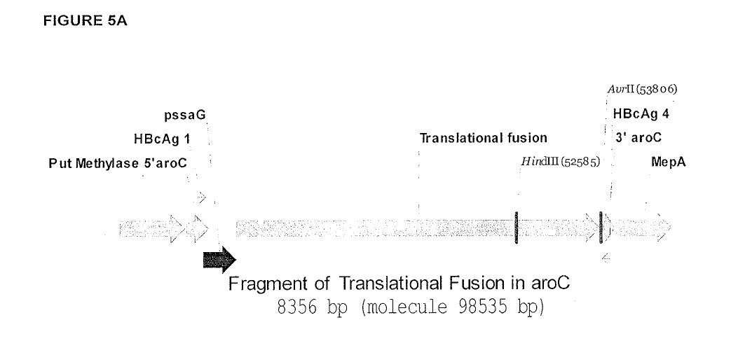

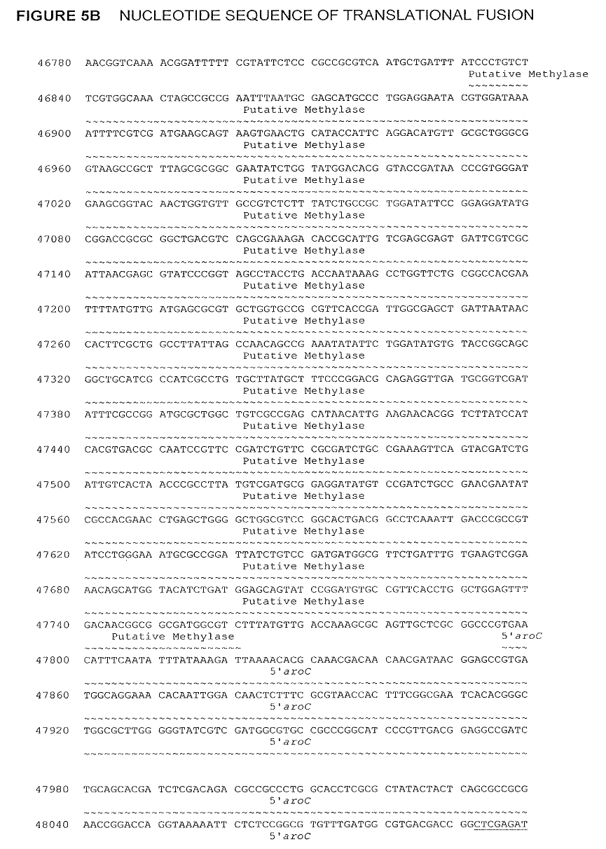

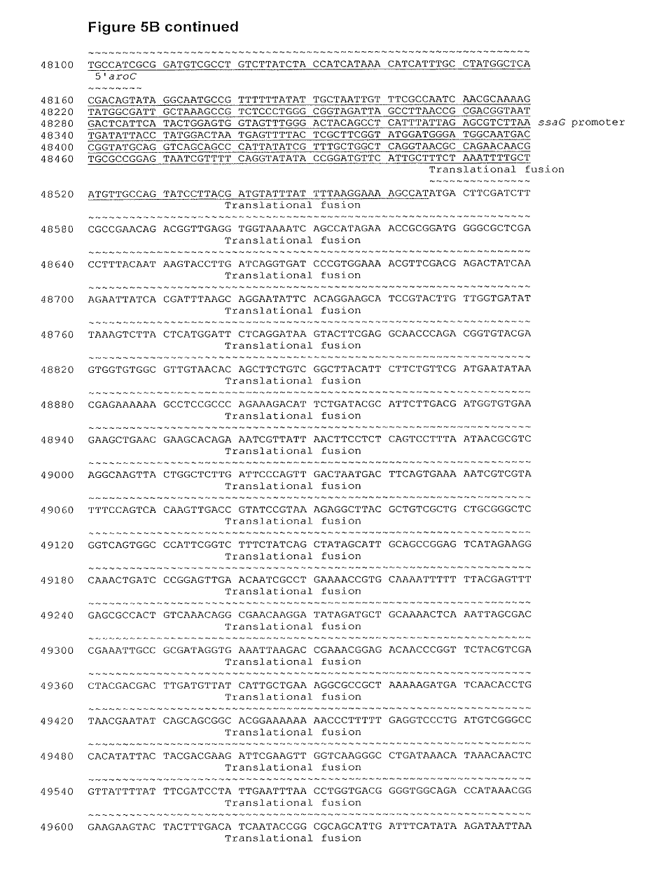

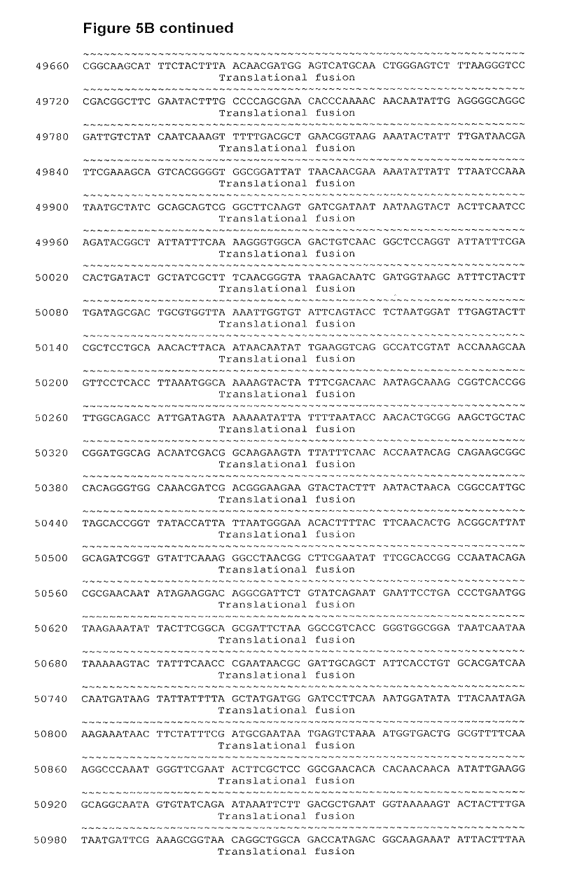

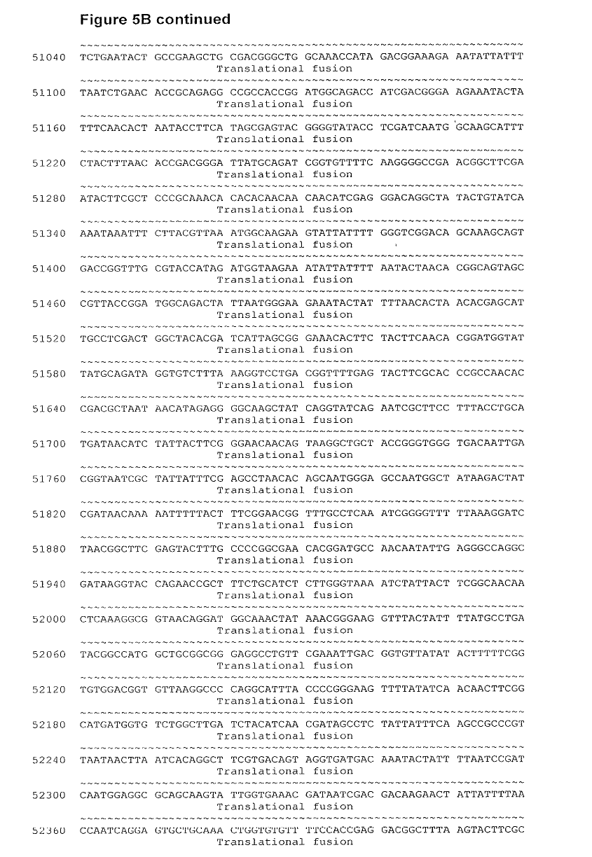

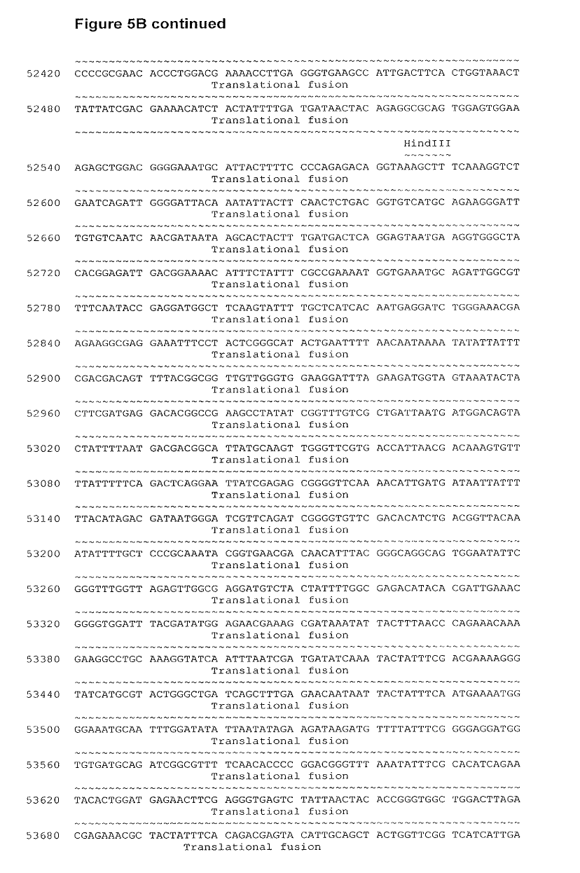

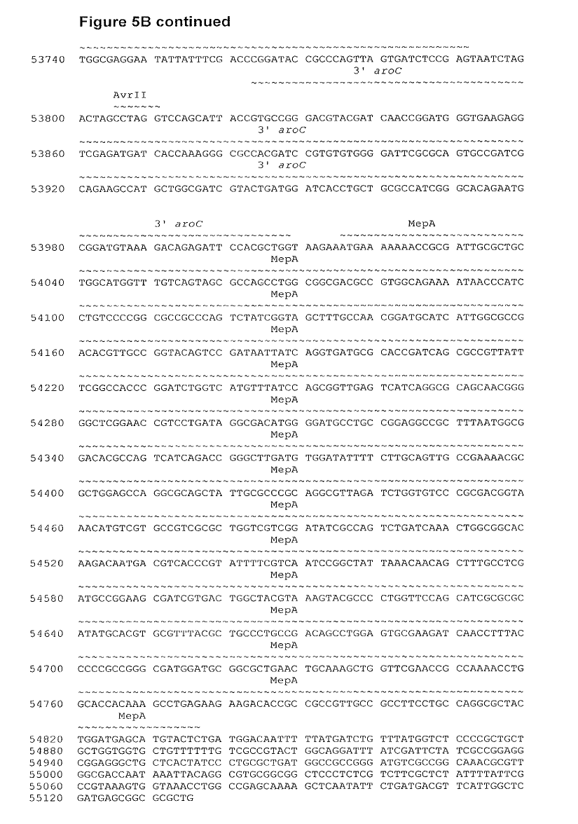

A second vaccine strain was designed to express a translational fusion of clyA-toxin A C-terminal repeat toxin B C-terminal repeat (FIG. 5C), shown diagrammatically in FIG. 5A, under the control of the ssaG promoter. The nucleotide and amino acid sequences of the translational fusion are shown in FIGS. 5B and 5C, respectively. The polynucleotide encoding the translational fusion is inserted at the aroC gene deletion site of S. typhi ZH9 strain.

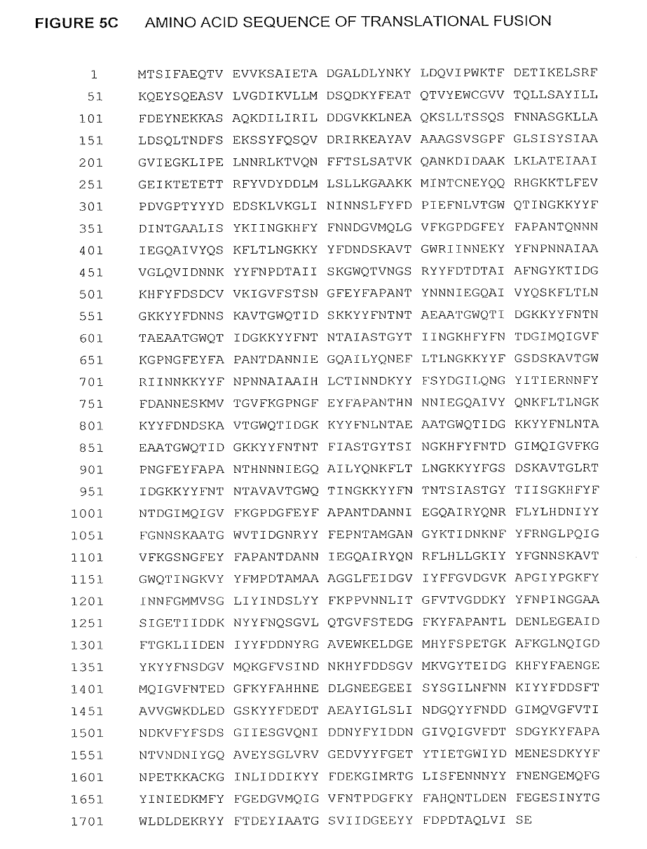

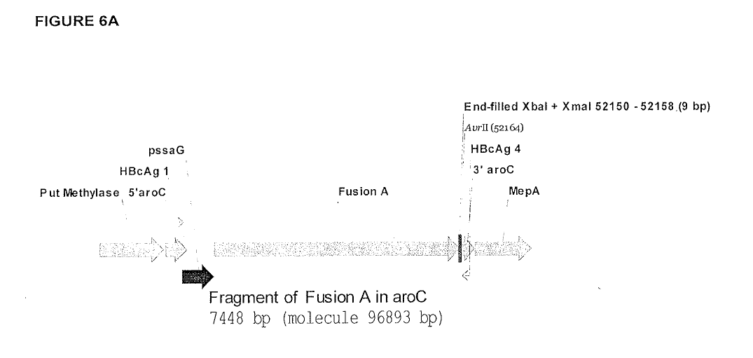

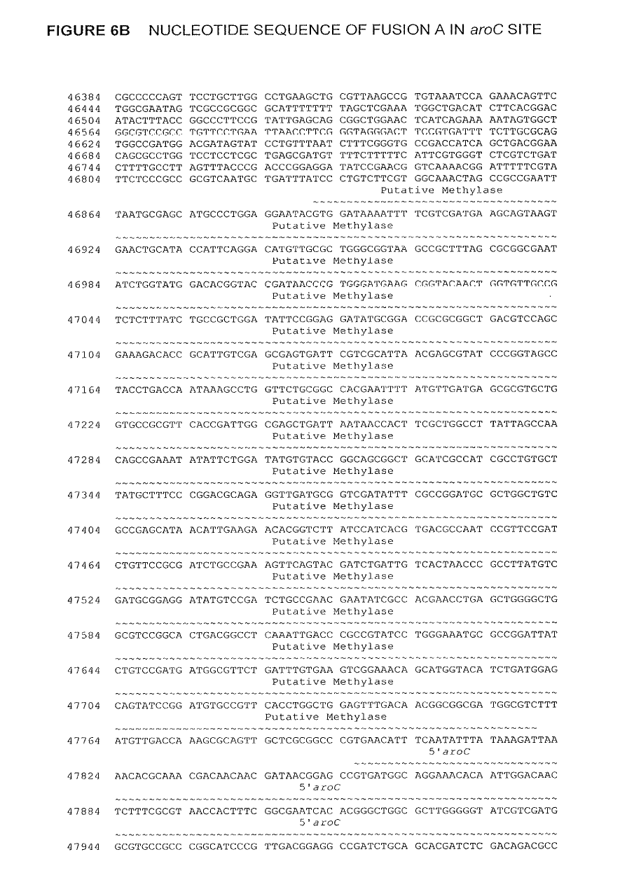

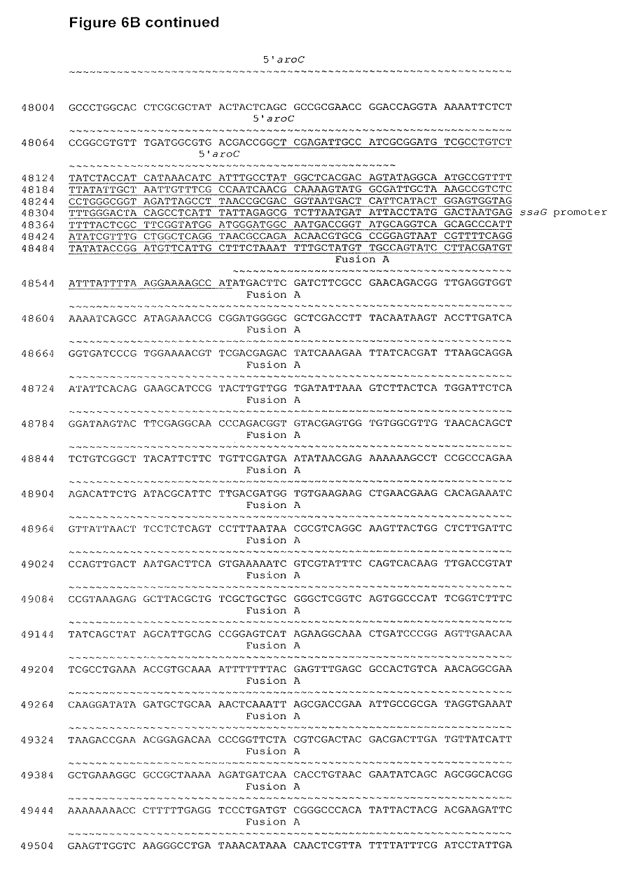

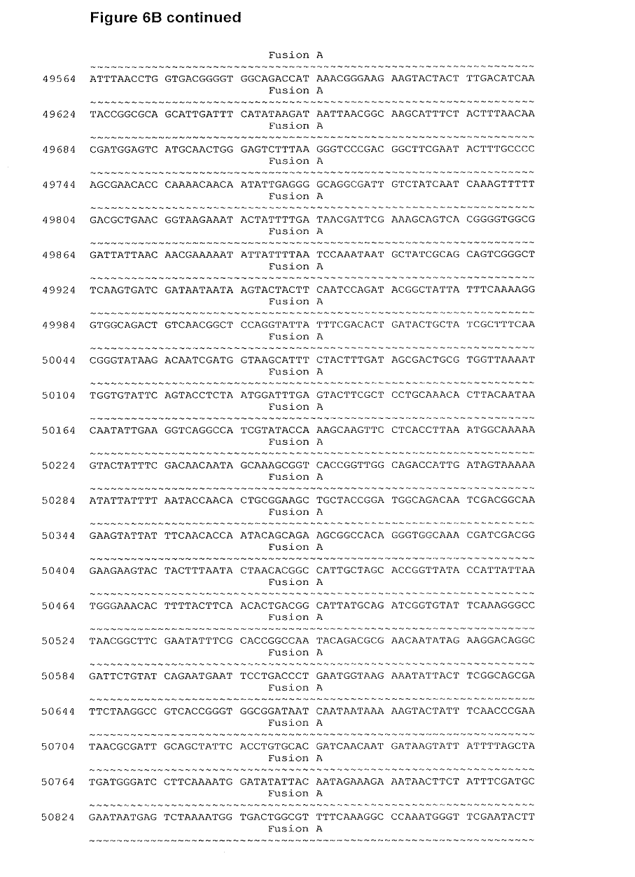

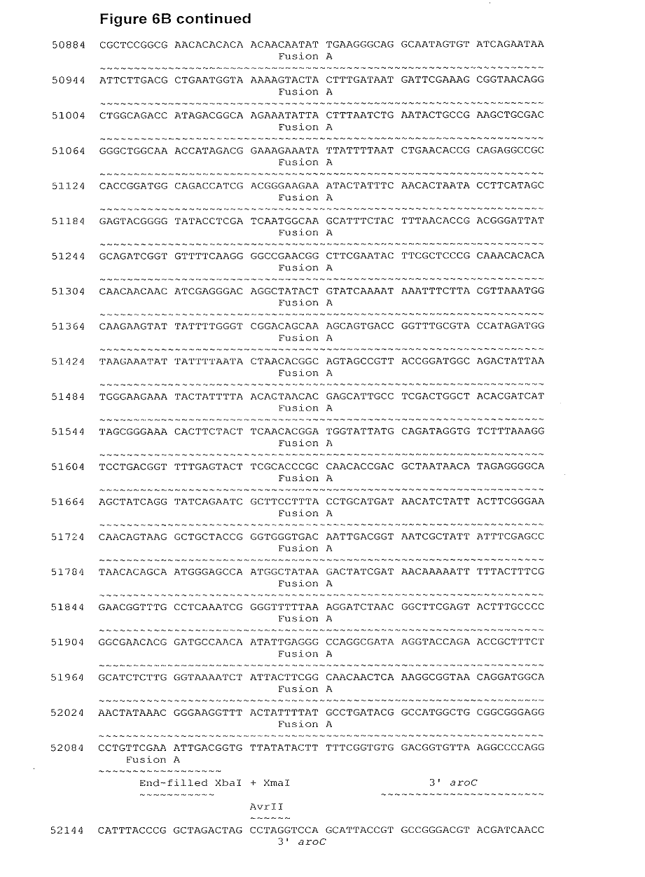

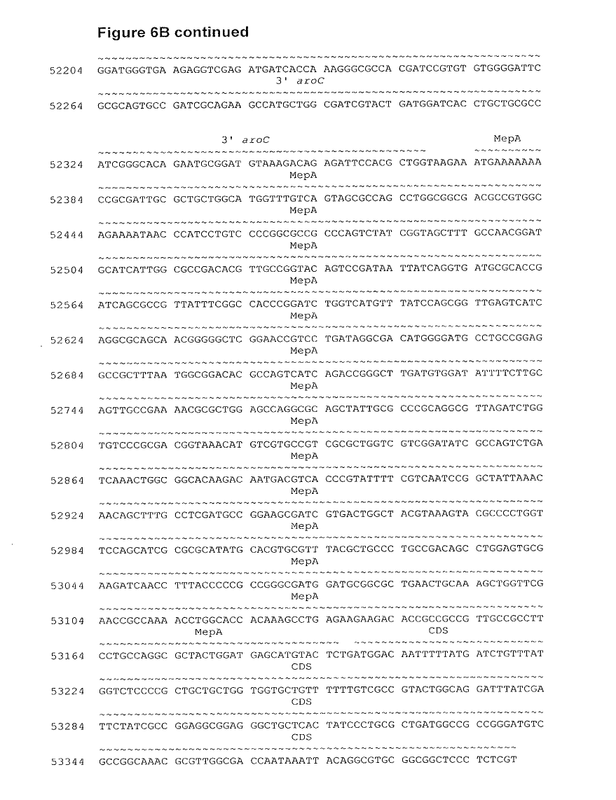

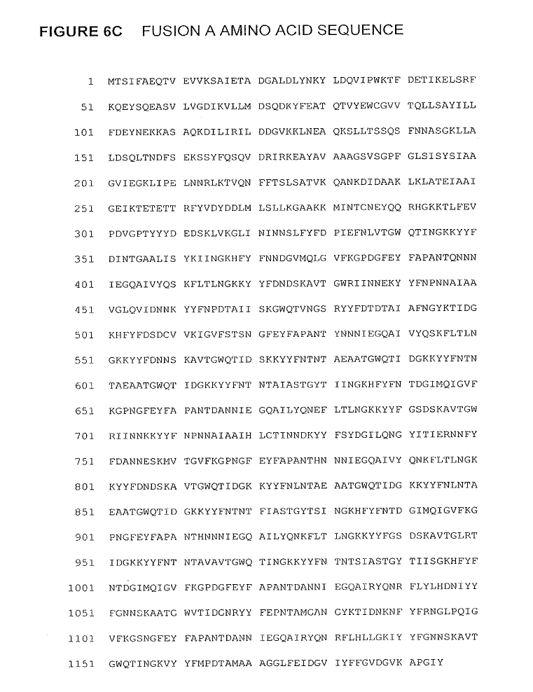

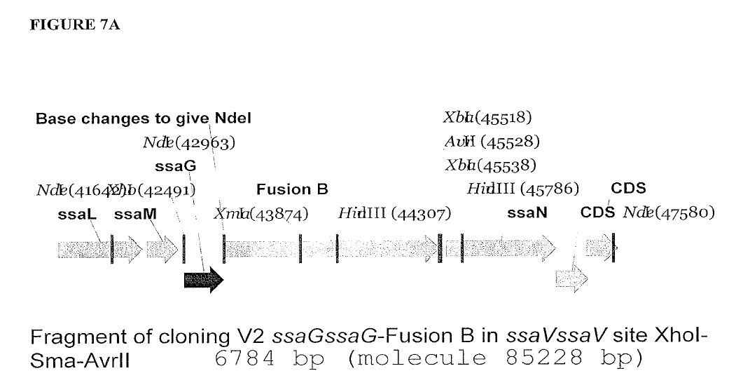

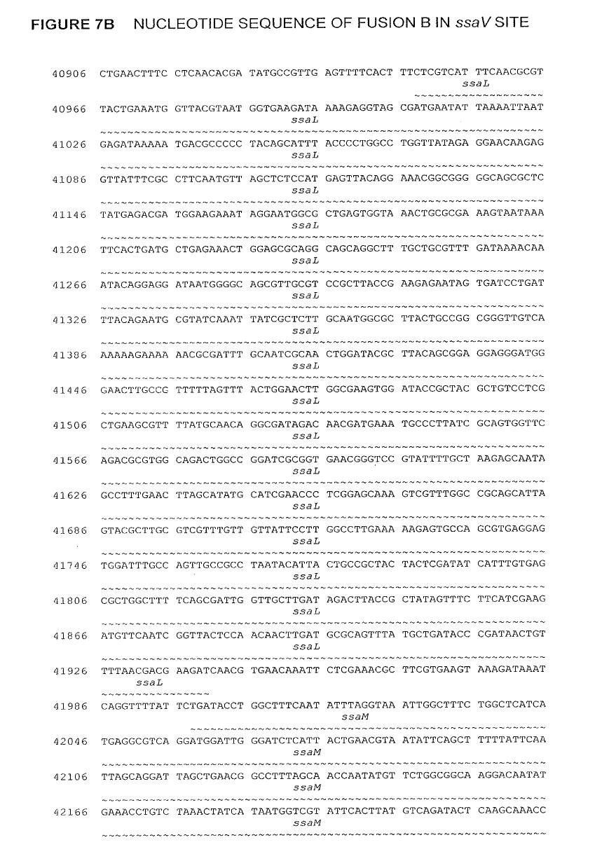

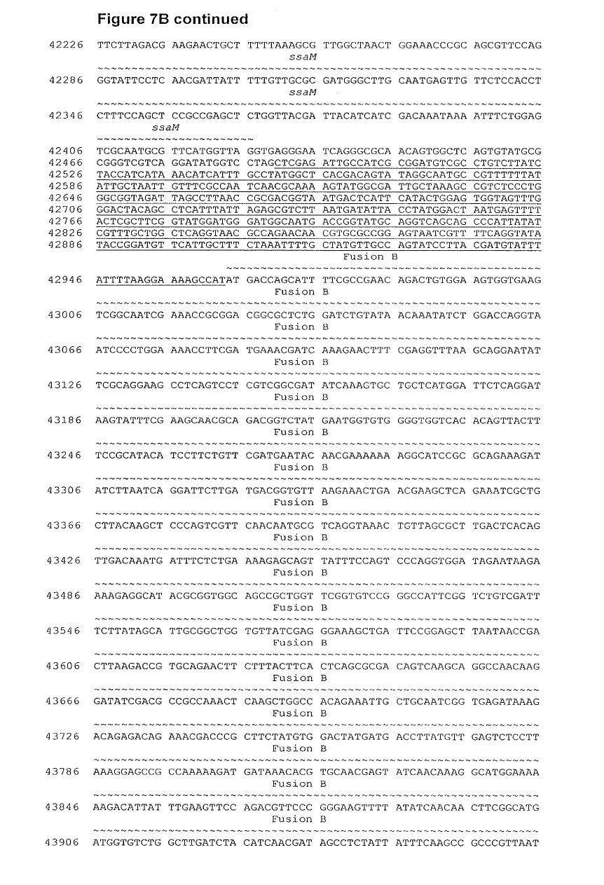

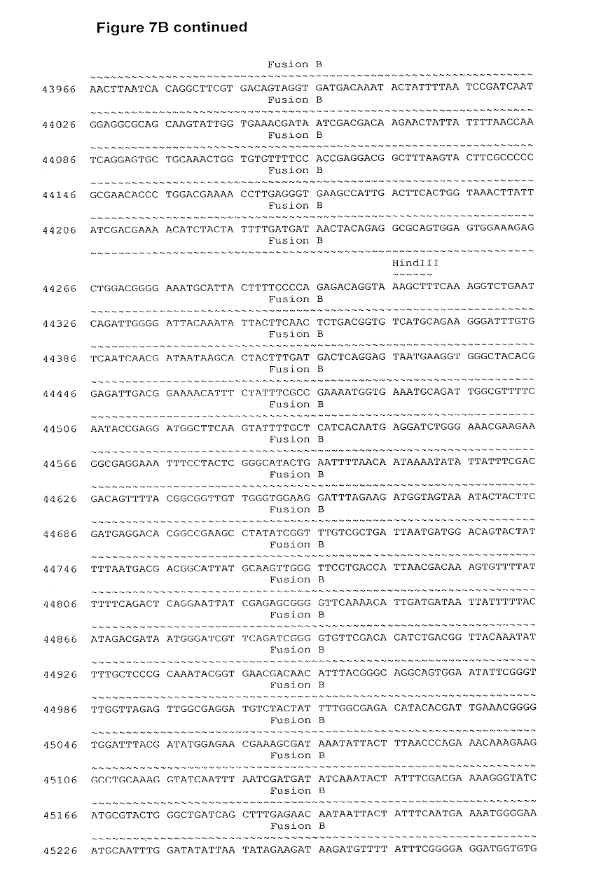

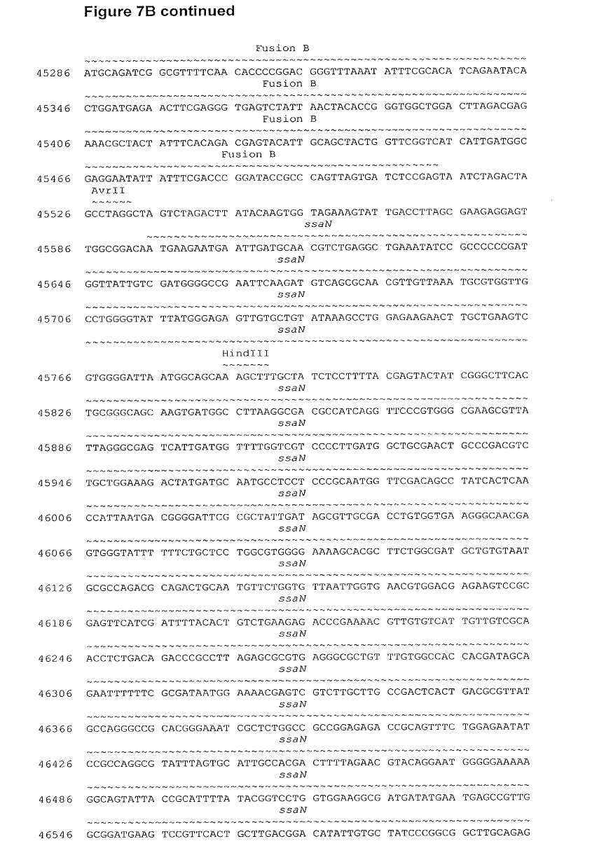

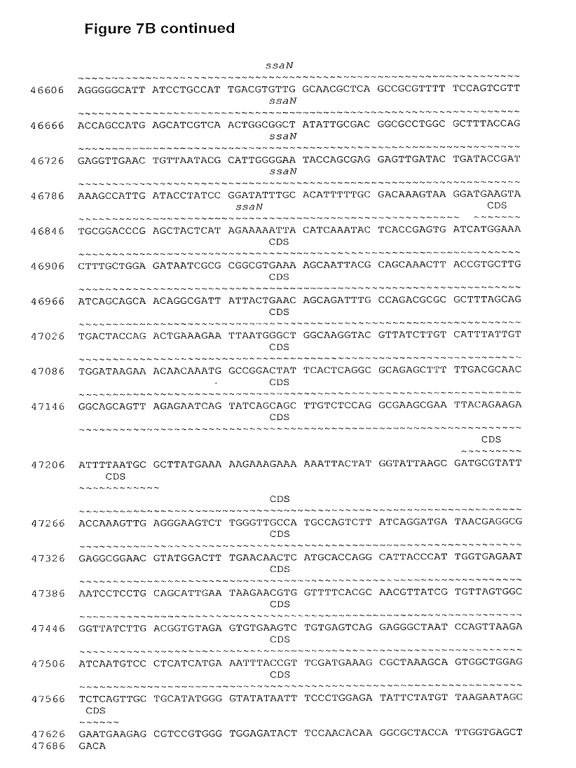

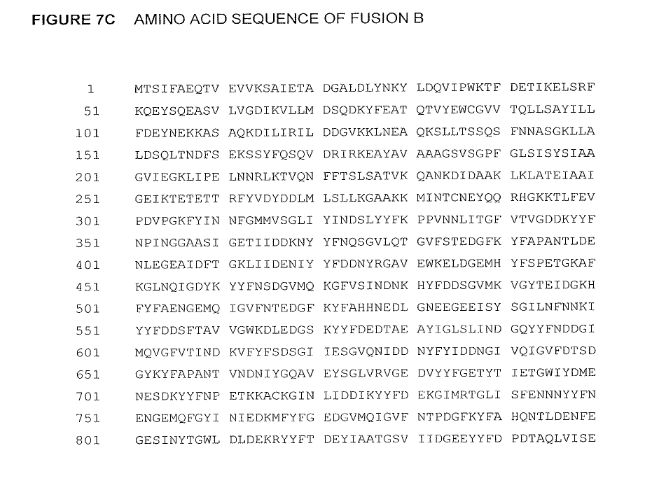

A third and fourth vaccine strains were designed to express Fusion A (FIG. 6C) and Fusion B (FIG. 7C), each under the control of a separate ssaG promoter. The third vaccine strain contains an insertion of the polynucleotide encoding Fusion A (shown in FIGS. 6A and 6B) at the aroC gene deletion site of S. typhi ZH9 strain. The third vaccine strain further contains an insertion of the polynucleotide encoding Fusion B (shown in FIGS. 7A and 7B) at the ssaV gene deletion site of S. typhi ZH9 strain. The fourth vaccine strain (LC5117) contains an insertion of the polynucleotide encoding Fusion A at the ssaV deletion site of S. typhi ZH9 strain. The fourth vaccine strain further contains an insertion of the polynucleotide encoding Fusion B at the aroC gene deletion site of S. typhi ZH9 strain.

TABLE 1
Summary of Vaccine Strains

| Strain | Genotype | Description |
|---|---|---|
| (1) | S. Typhi ZH9; | Transcriptional fusion at aroC site in S. Typhi |
| LC219 | Insertion at aroC region; | aroC::ssaG promoter - clyA - toxin A C- |
| | FusionA-FusionB | terminal repeat - clyA - toxin B C-terminal |
| | | repeat |
| | | ssaV- |
| (2) | S. Typhi ZH9; | Translational fusion at aroC site in S. Typhi |
| ZS121 | Insertion aroC region; | aroC::ssaG promoter - clyA - toxin A C- |
| | FusionAB | terminal repeat - toxin B C-terminal repeat |
| | | ssaV- |
| (3) | S. Typhi ZH9 | ssaV::ssaG promoter - clyA - toxin B C- |
| | Insertion at aroC region | terminal repeat |
| | (FusionA); | aroC::ssaG promoter - clyA - toxin A C- |
| | Insertion at ssaV region | terminal repeat |
| | (FusionB) | |
| (4) | S. Typhi ZH9 | ssaV::ssaG promoter - clyA - toxin A C- |
| LC5117 | Insertion at ssaV region | terminal repeat |
| | (Fusion A) | aroC::ssaG promoter - clyA - toxin B C- |
| | Insertion at aroC region | terminal repeat |
| | (Fusion B) | |

The promoter and coding DNA sequences may be cloned and prepared by conventional techniques known in the art. The encoding polynucleotides may be cloned directly into a suicide vector that has been modified to carry the flanking regions of the aroC deletion of host strain. An exemplary suicide vector for insertion of the transcriptional fusion shown in FIG. 3 at the aroC gene deletion site of S. Typhi ZH9.

### Example 2

### Determination of Toxin A and Toxin B C-Terminal Repeat Domain mRNA Levels in Strains

Three candidate spi-VEC C. difficile vaccine strains from Example 1 along with a ZH9 negative control (parent strain) were grown overnight at 37° C. with shaking in mod LB medium supplemented with aromatic compounds and tyrosine. Cells were then subcultured and grown to mid log phase. The cells were then collected by centrifugation and washed twice with LPM (low phosphate low magnesium) medium, pH7.0. The cells were then re-suspended in LPM medium at either pH5.8 or pH7 and incubated overnight at 37° C. with shaking. Media at pH 5.8 is designed to replicate the intracellular environment required to induce the ssaG promoter. Cell pellets were then collected and RNA extracted using the Ambion Ribopure bacteria kit, according to the manufactures instructions with inclusion of the optional DNaseI treatment step to remove contaminating DNA from the sample.

Each RNA sample was used as the template in three different Taqman RT− QPCR assays, performed using an ABI stepone instrument. The first assay determines the level of gyrB mRNA, this is an endogenous control which is used to normalise the signals seen in the other assays to account for variations in the amount of RNA recovered in each test sample. The second and third assays are designed to determine the levels of mRNA encoding the toxin and toxin B C-terminal repeat domains (antigen sequences). For each sample of RNA in each assay a no reverse transcriptase control was included. As in these controls no cDNA is generated from the RNA any amplification observed due to the carry over of genomic DNA. The relative RNA levels for each sample are then calculated using the following method:

Step 1 Normalisation to Endogenous gyrB Control

Ct_{tox assay}−Ct_{gyrB assay}=ΔCt

where
Ct_{tox assay}=threshold cycle for a sample in the toxin A or B assay
Ct_{gyrB assay}=threshold cycle for the same sample in the gyrB assay
ΔCt=relative threshold cycle

Each cell contains a consistent number of gyrB mRNA molecules, this step therefore corrects for any variation in the extraction efficiency and number of cells used, for each extraction

### Step 2 Normalisation to RT− Sample

ΔCt_{RT+}−ΔCt_{RT-}=ΔΔCt

where
=relative CT value for the sample for the reaction containing RTase
=relative CT value for the same sample for the reaction without RTase

The amplification seen in the RT+ wells is a combination of amplification of cDNA and carried over genomic DNA. The amplification seen in the RT− wells is only due to amplification of carried over genomic DNA. The ΔΔCt therefore corresponds to the relatiave CT value for amplification of cDNA.

### Step 3 Transformation

relative value=2^{-ΔΔCt}

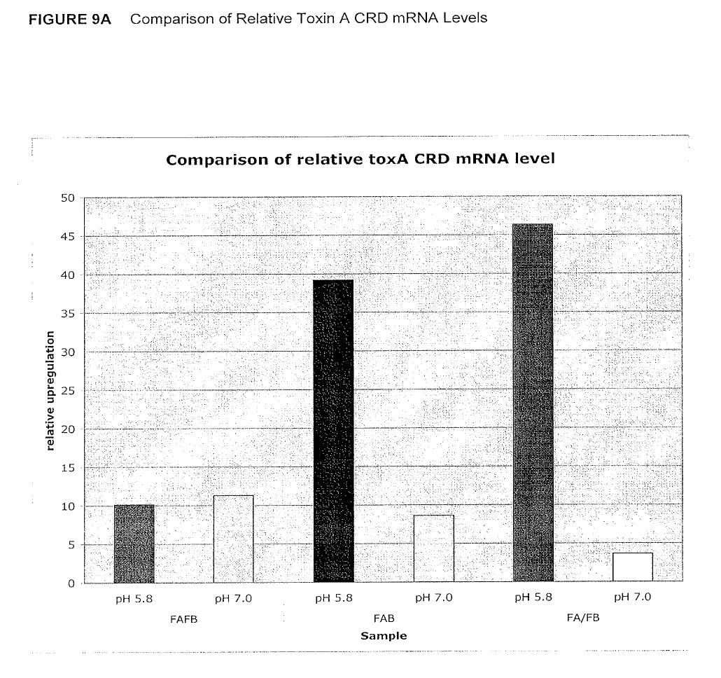

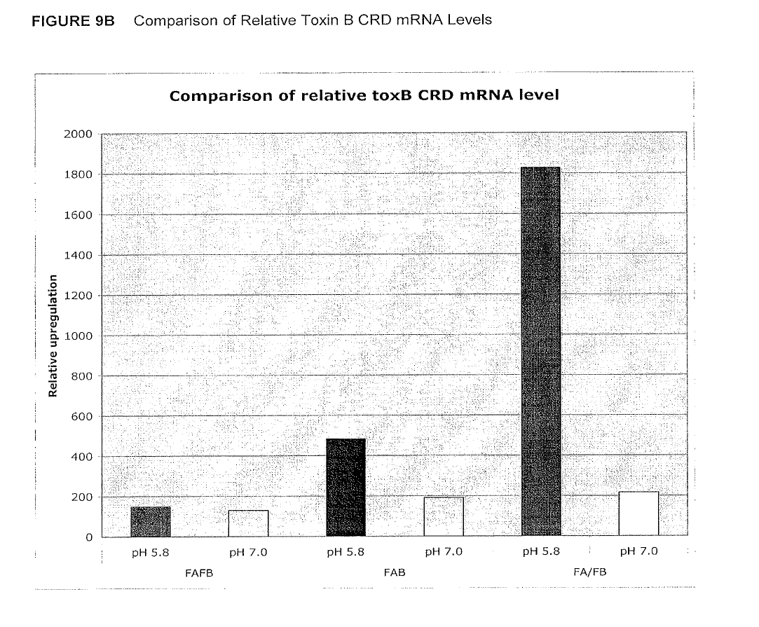

As expected, the ZS121 and LC5117 strains showed increased mRNA levels for both antigens at pH 5.8 compared to pH 7.0 (see Table 2 below and FIGS. 9A and 9B). The LC219 strain did not show the expected upregulation on reduction in pH.

TABLE 2
RT-QPCR Results

| | Strain | | | | | |
|---|---|---|---|---|---|---|
| | FAFB (LC219) | | FAB (ZS121) | | FA/FB (LC5117) | |
| | growth condition | | | | | |
| | pH 5.8 | pH 7.0 | pH 5.8 | pH 7.0 | pH 5.8 | pH 7.0 |
| relative value | 10.15982 | 11.32457 | 39.20493 | 8.653748 | 46.37718 | 3.658475 |
| toxin A mRNA | | | | | | |
| relative value | 149.1205 | 130.5054 | 484.411 | 191.3278 | 1827.886 | 216.9019 |
| toxin B mRNA | | | | | | |

### Example 3

### Mouse Challenge Study

Female Balb/C mice will be tested for development of antibody immunity to C. difficile toxins A and B after administration of 3 of the spi-VEC constructs provided in Example 1. The 3 spi-VEC constructs and control that will be utilized are:

1) S. typhi (Ty2 aroC::FAFB ssaV−); strain LC219

2) S. typhi (Ty2 aroC::FAB ssaV−); strain ZS121

3) S. typhi (Ty2 aroC::FB ssaV::FA); strain LC5117

4) ZH9 (empty spi-VEC strain)

Three immunizations will be given to each test or control groups on days 0, 21 and 42. Each group of mice will contain 10 mice for a total of 140 mice. The vaccines will be administered intranasally, subcutaneously or orally depending on group. The Table 3 provides a description of the test groups.

TABLE 3
Experimental Groups

| | | Delivery | |
|---|---|---|---|
| Group | Strain | day 0, d 21, d 42 | Dose level |
| 1 | S. typhi Ty2 | intranasal 2 × 25 mcL | 10e8 or TBD |
| 2 | LC219 | intranasal 2 × 25 mcL | 10e8 or TBD |
| 3 | ZS121 | intranasal 2 × 25 mcL | 10e8 or TBD |
| 4 | LC5117 | intranasal 2 × 25 mcL | 10e8 or TBD |
| 5 | S. typhi Ty2 | subcutaneous 2 × 100 mcL | 10e8 or TBD |
| 6 | LC219 | subcutaneous 2 × 100 mcL | 10e9 or TBD |
| 7 | ZS121 | subcutaneous 2 × 100 mcL | 10e9 or TBD |
| 8 | LC5117 | subcutaneous 2 × 100 mcL | 10e9 or TBD |
| 9 | None | None | None |
| 10 | None | CRD A protein | 2.5 mcg on 125 mcg alum |
| 11 | LC5117 | Prime-Boost: | 10e9 bacteria; |
| | | intranasal 2 × 25 mcL on day 0; boosted | Toxoid A or CRDA at 2.5 mcg on |
| | | with protein (either toxoid A or CRDA) | 125 mcg alum |
| | | on days 21 and 42 | |
| 12 | S. typhi Ty2 and | Vector Immunity: Ty2 intranasal | 10e9 |
| | LC5117 | 2 × 25 mcL on day 0; boosted with | |
| | | LC5117 intranasal 2 × 25 mcL on days | |
| | | 21 and 42 | |
| 13 | S. typhi Ty2 | intranasal 2 × 25 mcL | 10e9 |
| | with | | |
| | aroC::Chlamydia | | |
| | CT84 ssaV- (no | | |
| | cly A) | | |
| 14 | LC5117 | oral | 10e9 |

Serum samples will be obtained prior to experimentation (prebleed) and about at days 18, 39 and 56 for all mice. From 5 mice of groups 1, 5 and 9, serum samples will be obtained on day 1, within 24 hours after administration of bacteria. From another 5 mice of groups 1, 5 and 9, serum samples will be obtained on day 4 after administration of bacteria. Briefly, sera will be obtained from designated mice using a glass micropipette from the orbital plexus into a Microtainer, allowed to clot, centrifuged and serum fraction obtained.

Collected sera can be used for various ELISA assays. For instance, an ELISA utilizing a FITC BSA plate coat can be used to determine serum IgM or serum IgG antibody increases above background. In this example, because fluorescein is an irrelevant antigen, significant levels of serum anti-FITC-BSA will be indicative of polyclonal activation. Other ELISAs that may be used are ones which measure TN F-alpha content, anti-CRD A (e.g., CRD A plate coat), anti-CRD B (e.g., CRD B plate coat), anti-C. difficile toxoid A and anti-C. difficile toxoid B.

Fecal pellets will also be collected and analyzed by ELISA. Briefly, fecal pellets will be collected at day −2 or −1, day 9, day 30 and day 51. Fresh fecal pellets will be collected by placing a mouse in a clean cage with appropriate lining (clean and sterile paper towel or bedding) in order to obtain two fecal pellets per mouse and 10 fecal pellets per group. Using clean foreceps, pellets will be placed in sterile tubes and stored on ice. Within one hour of storage, 1 mL of PBS with 0.05% BSA, 0.05% azide and 100 μg/mL thimerosal will be added to each tube and vortexed for about 30 seconds. The tubes will then be incubated at 4° C. for 30 minutes, vortexed again for about 30 seconds, incubated again at 4° C. for 30 minutes, and then subjected to constant mechanical agitation for 1 hour at 4° C. Tubes will then be centrifuged for 5 minutes at 1000×g. Supernatant will be removed and stored frozen until assayed using ELISA.

On about day 60, surviving mice will be humanely euthanized. Spleens from five mice of each group may be removed for assaying. In particular, it is envisioned that the incease in proliferation of cultured splenocytes due to recall antigen (as fold increase over background) may be determined. Also, IFN-gamma concentration in supernatants of cultured splenocytes may be determined. Further, a determination of increased frequency of IFN-gamma producing cells using ELISPOT technique and comparison to un-immunized mice may be performed.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1-39. (canceled)

40. An attenuated microorganism expressing an immunogenic peptide, the immunogenic peptide comprising about 15 to about 20 repeat regions of a Clostridium difficile Toxin A C-terminal repeat region and about 15 to about 24 repeat regions of a C. difficile Toxin B C-terminal repeat region, wherein said microorganism induces an effective immune response against said immunogenic peptide when administered to a human patient.

41. The attenuated microorganism of claim 40, wherein the microorganism is an attenuated Salmonella comprising a gene expression cassette that directs the expression of the immunogenic peptide from an inducible promoter.

42. The attenuated microorganism of claim 40, wherein the immunogenic peptide is secreted from the microorganism via a secretion signal.

43. The microorganism of claim 40, wherein said microorganism induces mucosal immunity against said immunogenic peptide when orally administered to the patient.

44. The microorganism of claim 40, wherein the microorganism is an attenuated Salmonella having a deletion or inactivation of a gene involved in the biosynthesis of aromatic compounds and a deletion or inactivation of a gene encoded on the Salmonella pathogenicity island 2 (SPI-2).

45. The microorganism of claim 44, wherein the gene involved in the biosynthesis of aromatic compounds is aroC.

46. The microorganism of claim 44, wherein the gene encoded on SPI-2 is ssaV.

47. The microorganism of claim 44, wherein the attenuated Salmonella microorganism is derived from Salmonella enterica serovar Typhi ZH9.

48. The microorganism of claim 40, wherein the about 15 to about 20 repeats of the C. difficile Toxin A C-terminal repeat region and the about 15 to about 24 repeats of the C. difficile Toxin B C-terminal repeat region are secreted via a ClyA secretion signal of a non-hemolytic derivative thereof.

49. The microorganism of claim 40, wherein the polynucleotide encoding the about 15 to about 20 repeats of the C. difficile Toxin A C-terminal repeat region and the about 15 to about 24 repeats of the C. difficile Toxin B C-terminal repeat region contains codons optimized for gene expression in Salmonella.

50. The microorganism of claim 49, wherein the polynucleotide has a G/C content of about 50%.

51. The microorganism of claim 40, wherein expression of the about 15 to about 20 repeat units of the C. difficile Toxin A C-terminal repeat region and about 15 to about 24 repeat units of the C. difficile Toxin B C-terminal repeat region are controlled by a Salmonella ssaG promoter.

52. A composition comprising the microorganism of claim 40, and a pharmaceutically acceptable carrier and/or diluent.

53. The composition of claim 52, further comprising at least one adjuvant.

54. The microorganism of claim 44, wherein the gene involved in the biosynthesis of aromatic compounds is aroC and the gene encoded on SPI-2 is ssaV.

55. The microorganism of claim 40, comprising about 19 repeat units of a C. difficile Toxin A C-terminal repeat region and about 24 repeat units of a C. difficile Toxin B C-terminal repeat region.

56. An attenuated Salmonella microorganism suitable for vaccination against C. difficile, the microorganism comprising a gene expression cassette directing the expression of an immunogenic peptide from a Salmonella ssaG promoter, wherein the immunogenic peptide comprises about 15 to about 20 repeat units of a C. difficile Toxin A C-terminal repeat region, and about 15 to about 24 repeat units of a C. difficile Toxin B C-terminal repeat region.

57. The microorganism of claim 56, wherein the microorganism is an attenuated Salmonella having a deletion or inactivation of a gene involved in the biosynthesis of aromatic compounds and a deletion or inactivation of a gene encoded on the Salmonella pathogenicity island 2 (SPI-2), wherein the gene involved in the biosynthesis of aromatic compounds is aroC and the gene encoded on SPI-2 is ssaV.

58. The microorganism of claim 56, comprising about 19 repeat units of a C. difficile Toxin A C-terminal repeat region and about 24 repeat units of a C. difficile Toxin B C-terminal repeat region.

59. An attenuated microorganism expressing an immunogenic peptide, the immunogenic peptide consisting of an immunogenic portion of a C. difficile Toxin A C-terminal repeat region and C. difficile Toxin B C-terminal repeat region, wherein said microorganism induces an effective immune response against said immunogenic peptide when administered to a human patient.
